SEQUENCE LISTING (17 sequences)

SEQ ID NO: 1 - protein, 46 aa, Homo sapiens
EPTIWFGKGHSGMLASEGREAVLTRLHESERVRKQERERDTEERRE

SEQ ID NO: 2 - protein, 23 aa, Homo sapiens
ASFELLGSSNPPASASQSAGIIG

SEQ ID NO: 3 - protein, 16 aa, Homo sapiens
GEHLLNSGFEAWKPSP

SEQ ID NO: 4 - protein, 46 aa, Homo sapiens
DPCKLKEEITSDIESPYKTEVTKGQAEVCESVCAYVYQFFLQVKQD

SEQ ID NO: 5 - protein, 36 aa, Homo sapiens
DATVVLLKKGRKEGIRKEEWMDGWIANFHRKASVIM

SEQ ID NO: 6 - protein, 40 aa, Homo sapiens
WAMYAKFDQYSRGRGLQFVVGGGSGGGWLWYTRNLVDQGN

SEQ ID NO: 7 - protein, 14 aa, Homo sapiens
CTELRVRDLWMLCV

SEQ ID NO: 8 - protein, 12 aa, Homo sapiens
CVPGSAFTDMFL

SEQ ID NO: 9 - protein, 15 aa, Homo sapiens
PGLLWELGSGRLSGD

SEQ ID NO: 10 - protein, 95 aa, Homo sapiens
QPRAGPSHSRSPRSSSRPSSLSAWCQLHRLRMPSPHSLALVQPAGHGLQRDDCVGVTEPACCAEVCEWAWAHTCKHALANLSTQCACVDVCVCKG

SEQ ID NO: 11 - protein, 18 aa, Homo sapiens
RPAPPVRVNCHVDFVLSF

SEQ ID NO: 12 - protein, 29 aa, Homo sapiens
ECSGMITAHCSLNFLGSSDPLPSSWDYRL

SEQ ID NO: 13 - protein, 29 aa, Homo sapiens
VHSDTCDLDKQKWAGETLEKRSSDGLELC

SEQ ID NO: 14 - protein, 29 aa, Homo sapiens
SFSASFCMGGFGHSLSIFESLLCAGHSAR

SEQ ID NO: 15 - protein, 30 aa, Homo sapiens
VRAPRRGPLGGRKKKKAPSASDSDSKADSD

SEQ ID NO: 16 - protein, 73 aa, Homo sapiens
VRAPRRGPLGGRKKKEPTIWFGKGHSGMLASEGREAVLTRLHESERVKQERDTEERREKAPSASDSDSKADSD

SEQ ID NO: 17 - protein, 10 aa, synthetic construct
GGGSHHHHHH

(19) United States
(12) Patent Application Publication
(10) Pub. No.: US 2025/0355002 A1
(43) Pub. Date: Nov. 20, 2025

(54) IDENTIFICATION OF TDP-43 CRYPTIC EXON-ENCODED NEOEPITOPES AS FUNCTIONAL FLUID BIOMARKERS FOR ALZHEIMER'S DISEASE AND RELATED DEMENTIA

(71) Applicant: The Johns Hopkins University, Baltimore, MD (US)

(72) Inventors: Philip C. Wong, Baltimore, MD (US); Jonathan P. Ling, Baltimore, MD (US)

(21) Appl. No.: 18/871,998

(22) PCT Filed: Jun. 14, 2023
(86) PCT No.: PCT/US2023/068441

§ 371 Date: Dec. 5, 2024

Related U.S. Application Data
(60) Provisional application No. 63/352,113, filed on Jun. 14, 2022.

(51) Int. Cl. G01N 33/68; C07K 16/18

(52) U.S. Cl. CPC G01N 33/6896; C07K 16/18; G01N 33/6878; C07K 2317/34; G01N 2333/475; G01N 2800/2821; G01N 2800/2835; G01N 2800/285; G01N 2800/52

(57) ABSTRACT

The invention provides antibodies and binding fragments thereof that specifically binds to TDP-43 cryptic exon-encoded neoepitopes, and methods of use thereof. The methods of use include methods of detecting TDP-43 loss of function, methods of detection and/or diagnosing TDP-43 associated diseases, and methods of monitoring disease progression and/or response to therapy. The invention also provides a kit including the antibodies and binding fragments thereof.

### CROSS-REFERENCE TO RELATED APPLICATION

This application claims the benefit of priority under 35 U.S.C § 119 (e) of U.S. Provisional Patent Application No. 63/352,113, filed Jun. 14, 2022. The disclosure of the prior application is considered part of and is hereby incorporated by reference in its entirety.

### STATEMENT REGARDING FEDERALLY SPONSERED R&D

This invention was made with government support under Grant No. NS095969 awarded by the National Institutes of Health. The government has certain rights in the invention.

### INCORPORATION BY REFERENCE OF SEQUENCE LISTING

The material in the accompanying sequence listing is hereby incorporated by reference into this application. The accompanying sequence listing xml file, name Jun. 13, 2023-JHU4530-1WO_SL Sequence Listing ST26.xml, was created on Jun. 13, 2023 and is 16 kb.

### BACKGROUND OF THE INVENTION

### Field of the Invention

The present invention relates generally to Alzheimer's disease and related dementia, and more specifically to tools for detecting TDP-43 loss of function for the detection and monitoring of such diseases.

### Background Information

Tar DNA-binding protein 43 (TDP-43, encoded by the gene TARDBP), is a highly conserved RNA binding protein that is implicated in amyotrophic lateral sclerosis, a progressive neurodegenerative disease characterized by the death of upper and lower motor neurons. In nearly all cases of sporadic ALS, TDP-43 in motor neurons depletes from the nucleus and aggregates in the cytoplasm. Missense mutations in TDP-43, which mostly cluster within its C-terminal domain, are linked to familial ALS. Various other genetic mutations associated with familial ALS are also associated with TDP-43 pathology, supporting the notion that TDP-43 mis-localization is central to its pathogenesis. In addition, TDP-43 pathology is evident in cases with frontotemporal dementia, inclusion body myositis, and Alzheimer's disease.

The loss of TDP-43 function plays a critical role in motor neuron degeneration as it plays important roles in several cellular processes, including cellular stress response pathways, mRNA delivery to dendritic or axonal compartments, or phase separation of membrane-less organelles. As a member of the heterogeneous ribonuclear protein (hnRNP) family, nuclear TDP-43 is concentrated in transcriptionally active euchromatin regions and regulate alternative splicing. TDP-43 interacts with many proteins and RNAs, potentially regulating numerous pathways. TDP-43 acts as a guardian of the transcriptome by repressing the splicing of non-conserved, unannotated ‘cryptic’ exons, a function that is compromised in cases of ALS and other neurodegenerative diseases with TDP-43 pathology. TDP-43-mediated splicing repression is central to the physiology of motor neurons, however, there is still an unmet need for tools for detecting TDP-43 mediated splicing repression during early stages, including pre-symptomatic phase, of illness.

### SUMMARY OF THE INVENTION

The present invention is based on the seminal discovery that TDP-43 loss of function results in the loss of cryptic exon splicing which generates neoepitopes, and more specifically to the development of antibodies and binding fragments thereof that specifically bind to the neoepitopes.

In one embodiment, the invention provides a method of detecting TDP-43 loss of function in a subject including contacting a sample from the subject with an antibody or binding fragment thereof which specifically binds to a cryptic exon-encoded neoepitope, wherein the cryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2), thereby detecting TDP-43 loss of function in the subject.

In one aspect, detecting TDP-43 loss of function includes detecting cryptic exon-encoded neoepitope in the sample from the subject. In some aspects, the sample is a biological fluid. In various aspects, the biological fluid is selected from the group consisting of blood, cerebrospinal fluid (CSF), saliva, sputum, urine or another biofluid. In one aspect, the cryptic exon-encoded neoepitope is within HDGFL2.

In another embodiment, the invention provides a method of detecting and/or diagnosing a TDP-43-associated disease in a subject including detecting TDP-43 loss of function in the subject, wherein detecting TDP-43 loss of function in the subject includes contacting a sample from the subject with an antibody or binding fragment thereof which specifically binds to a cryptic exon-encoded neoepitope, thereby detecting or diagnosing the TDP-43-associated disease in the subject.

In one aspect, the method further includes detecting one or more additional TDP-43-associated biomarkers in the sample. In some aspects, the one or more TDP-43-associated biomarkers are selected from the group consisting of neurofilament (NF), tau, amyloid-β, α-synuclein, and combinations thereof. In other aspects, the TDP-43-associated disease is selected from the group consisting of Alzheimer's disease (AD), amyotrophic lateral sclerosis (ALS), frontotemporal lobar degeneration (FTLD), inclusion body myositis (IBM), primary age-related tauopathy (PART)/Neurofibrillary tangle-predominant senile dementia, chronic traumatic encephalopathy (CTE), progressive supranuclear palsy (PSP), corticobasal degeneration (CBD), frontotemporal dementia and parkinsonism linked to chromosome 17 (FTDP-17), lytico-bodig disease (Parkinson-dementia complex of Guam), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, chronic traumatic encephalopathy, limbic-predominant age-related TDP-43 encephalopathy (LATE), multiple sclerosis (MS) and TDP-43 encephalopathy. In various aspects, the TDP-43-associated disease is selected from the group consisting of AD, ALS, FTLD, IBM, CTE and MS. In some aspects, the TDP-43 associated disease is an early stage of the disease or in a pre-symptomatic phase of the disease.

In an additional embodiment, the invention provides a method of monitoring a TDP-43-associated disease progression and/or response to a TDP-43-associated disease therapy in a subject including detecting cryptic exon-encoded neoepitopes in a sample from the subject, wherein detecting cryptic exon-encoded neoepitopes in the sample includes contacting the sample with an antibody or binding fragment thereof which specifically binds to a cryptic exon-encoded neoepitope, thereby monitoring the TDP-43-associated disease progression and/or response to therapy in the subject.

In one aspect, detecting is repeated over time. In some aspects, an increase in the detection of a cryptic exon-encoded neoepitope or an increase in a number of cryptic exon-encoded neoepitopes detected in the sample in a first detection as compared to a second detection is indicative of disease progression and/or of an absence of response to the therapy. In other aspects, a decrease in the detection of a cryptic exon-encoded neoepitope or a decrease in a number of cryptic exon-encoded neoepitopes detected in the sample in a first detection as compared to a second detection is indicative of an absence of disease progression, a disease regression, and/or of a response to the therapy.

In a further embodiment, the invention provides a method of selecting a patient for enrollment in a clinical trial including detecting cryptic exon-encoded neoepitopes in a sample from the subject, wherein detecting cryptic exon-encoded neoepitopes in the sample includes contacting the sample with an antibody or binding fragment thereof which specifically binds to a cryptic exon-encoded neoepitope, thereby selecting the patient for enrollment in the clinical trial.

In one aspect, the clinical trial is investigating a therapy for the treatment of a TDP-43 associated disease. In some aspects, the TDP-43 associated disease is characterized by the expression of a cryptic exon-encoded neoepitope.

In one embodiment, the invention provides a method of predicting pheno-conversion of a TDP-43 associated disease in a subject including determining a ratio of a TDP-43-associated biomarker to a cryptic exon-encoded neoepitope in a sample from the subject, thereby predicting pheno-conversion in the subject.

In one aspect, the TDP-43-associated biomarker is selected from the group consisting of phosphorylated neurofilament heavy chain (pNFH), neurofilament light chain (NFL), tau, amyloid-β, α-synuclein, and combinations thereof. In another aspect, determining a ratio of pNFH to a cryptic exon-encoded neoepitope includes: (i) determining a level of cryptic exon-encoded neoepitope in a sample from the subject, and (ii) determining a level of phosphorylated neurofilament heavy chain in the sample from the subject. In another aspect, determining a level of cryptic exon-encoded neoepitope in a sample includes contacting the sample with an antibody or binding fragment thereof which specifically binds to a cryptic exon-encoded neoepitope. In various aspects, the cryptic exon-encoded neoepitope is HDGFL2. In one aspect, a ratio greater than 1 is indicative of a symptomatic stage of the TDP-43 associated disease. In another aspect, a ratio lesser than 1 is indicative of a pre-symptomatic stage of the TDP-43 associated disease. In some aspects, the TDP-43 associated disease is amyotrophic lateral sclerosis (ALS). In various aspects, the subject carries a C9ORF72 mutation.

In another embodiment, the invention provides a kit including a) one or more antibodies or binding fragment thereof which specifically bind to a cryptic exon-encoded neoepitopes; and b) instructions to use the antibodies of a) to detect TDP-43 loss of function in a sample, wherein the cryptic exon-encoded neoepitope is within (i) HDGFL2, (ii) ACTL6B, (iii) ARHGAP32, (iv) EPB41L4A, (v) SLC24A3, (vi) CDO1, (vii) AGRN, (viii) IGLON5, (ix) DNM1, (x) AARS1, (xi) PXDN, or (xii) NECAB2. In one aspect, the kit further includes an antibody or binding fragment thereof which specifically bind to phosphorylated neurofilament heavy chain (pNFH).

In an additional embodiment, the invention provides an antibody or binding fragment thereof which specifically binds to a cryptic exon-encoded neoepitope, wherein the cryptic exon-encoded neoepitope is an epitope resulting from a splicing incorporation of an exon normally repressed by TDP-43, and wherein the cryptic exon-encoded neoepitope is within (i) HDGFL2, (ii) ACTL6B, (iii) ARHGAP32, (iv) EPB41L4A, (v) SLC24A3, (vi) CDO1, (vii) AGRN, (viii) IGLON5, (ix) DNM1, (x) AARS1, (xi) PXDN, or (xii) NECAB2.

In one aspect, the splicing incorporation of an exon normally repressed by TDP-43 results from a TDP-43 loss of function. In another aspect, TDP-43 loss of function generates exons fused-in-frame with a translational reading frame to produce neoepitopes.

In one embodiment, the invention provides a method of detecting TDP-43 loss of function in a subject including detecting in a sample from the subject the presence of a cryptic exon-encoded neoepitope, wherein the cryptic exon-encoded neoepitope is an epitope resulting from a splicing incorporation of an exon normally repressed by TDP-43, and wherein the cryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2), thereby detecting TDP-43 loss of function in the subject.

In one aspect, the cryptic exon-encoded neoepitope is within HDGFL2. In another aspect, the sample is a biological fluid. In various aspects, the biological fluid is selected from the group consisting of blood, cerebrospinal fluid (CSF), saliva, sputum, urine or another biofluid. In one aspect, detecting the presence of a cryptic exon-encoded neoepitope is by enzyme-linked immunosorbent assay (ELISA), protein immunoprecipitation, immunoelectrophoresis, western blot, protein immunostaining, high-performance liquid chromatography (HPLC), or liquid chromatography-mass spectrometry (LC/MS).

### BRIEF DESCRIPTION OF THE DRAWINGS

FIG. 1 is a schematic representation of the strategy for developing cryptic peptide fluid biomarkers.

FIGS. 2A-2D show the identification of human in-frame TDP-43-associated cryptic exons. FIG. 2A shows UCSC genome browser visualization of selected cryptic exons in human motor neurons and HeLa cells aligned to the GRCh38 assembly. Arrows identify the non-conserved cryptic exons. Gene annotation below the RNA-sequencing tracks indicate the canonical exons (thick lines) and introns (thin lines). FIG. 2B shows a visualization of tissue-type specific gene expression of ACTL6B, AGRN, EPB41L4A, HDGFL2, and SLC24A3. FIG. 2C shows a comparison of wild-type (left) and cryptic (right) HDGFL2 protein structures. FIG. 2D shows the alignment of wild-type and cryptic HDGFL2 amino acid sequences.

FIGS. 3A-3B are photographs illustrating the generation of a series of antisera recognizing a cryptic exon-encoded protein in HDGFL2. FIG. 3A is a photograph of a immunoblot of protein lysates of HEK293 cells transfected with a construct expressing a GFP-myc-HDGFL2 fusion protein, subjected to protein blot analysis using antisera from seven monoclonal lines (1-65-1-71). FIG. 3B is an immunoblots of the same lysate as in FIG. 3A using anti-GFP antisera to confirm the identity of the GFP-myc-HDGFL2 fusion protein.

FIG. 4 is a photograph of an immunoblot illustrating the generation of a series of antisera recognizing a cryptic exon-encoded peptide in EPB41L4A. HEK293 cells were transfected with either GFP-myc-EPB41L4A (lanes 1-4, 6) or GFP alone (lanes 5, 7), and lysates were subjected to protein blot analysis using antisera from three monoclonal lines (22, 23, 24; lanes 1, 2, 3, respectively). Immunoblots of the same lysates using anti-myc antisera (lanes 4, 5) or anti-GFP antisera (lanes 6, 7) confirmed the identity of the GFP-myc-EPB41L4A fusion protein.

FIG. 5 is a photograph of agarose gel illustrating the identification of cryptic exons in siTDP HeLa. RT-PCR analysis of cryptic exons as indicated in Hela cells treated with siRNA against TDP-43. Circles indicate cryptic exons for HDGFL2, EPB41L4A, ATG4B, and GPSM2 present only in TDP-43-depleted HeLa cells.

FIGS. 6A-6C are photographs illustrating that the novel antibody shows specificity for HDGFL2 harboring cryptic exon-encoded peptide. FIG. 6A shows an immunoblot of total protein extracts of HeLa cells treated with control or TDP-43 siRNA (siTDP) subjected to protein blot analysis using an antibody specific to TDP-43. Note the marked reduction of TDP-43 in siTDP lane. FIG. 6B shows an immunoblot of total protein extracts as in FIG. 4A subjected to protein blot analysis using antibody against GAPDH as a control for total protein levels. FIG. 6C shows an immunoblot of total protein extracts as FIG. 6A subjected to protein blot analysis using either an antibody (Anti-CTB-50L17.10 antibody produced in rabbit) against the native HDGFL2 protein (left) or an antibody (1-69) recognizing the cryptic exon-encoded peptide within HDGFL2 (right). * denotes the novel HDGFL2 harboring the neo-epitope only when TDP-43 is depleted.

FIG. 7 is an immunoblot of the immunoprecipitation (IP) of cryptic HDGFL2; the IP-blot analysis was done in HeLa cells treated with siRNA against TDP-43 and control HeLa cells. IP with 1-69 antibody against cryptic HDGFL2. Probe for wild-type HDGFL2. * denotes the novel HDGFL2 harboring the neo-epitope only when TDP-43 is depleted.

FIGS. 8A-8D illustrate the development of an MSD assay specific for cryptic HDGFL2. FIG. 8A is a schematic representation of Sandwich ELISA using Meso Scale Discovery (MSD) system. FIG. 8B is a graph illustrating MSD assay detecting elevated cryptic HDGFL2 in siTDP HeLa lysate compared to control HeLa lysate. The starting total protein concentrations of both control and siTDP HeLa lysates were 4.5 ug/uL. They were diluted according to a 1/2 serial dilution series using 1× phosphate buffered saline. FIG. 8C is a graph illustrating that elevated siTDP MSD signal is specific to intact capture and detection antibodies. FIG. 8D is a graph illustrating average cryptic HDGFL2 signal elevated in CSF of C9ORF72 ALS compared to controls.

FIG. 9 is a graph illustrating MSD assay detecting elevated cryptic HDGFL2 in ALS CSF compared to control. FIG. 9 shows MSD detection of cryptic HDGFL2 in CSF of ALS individuals (C9orf72 mutation carriers) and control individuals. Duplicates of each CSF sample were assayed, and 4 wells were filled with blocking solution only (Buffer Only).

FIGS. 10A-10E show that cryptic HDGFL2 can be detected by MSD assay in CSF of both presymptomatic and symptomatic C9ORF72 mutation carriers. FIG. 10A is a schmetic representation of the original sandwich ELISA using Meso Scale Discovery (MSD) system. FIG. 10B is a graph illustrating elevated cryptic HDGFL2 detected in presymptomatic and symptomatic C9ORF72-mutation carriers compared to controls. FIG. 10C is a graph illustrating MSD detection of cryptic HDGFL2 in CSF of C9orf72 mutation carriers diagnosed with ALS (n=20), FTD (n=2), or ALS-FTD (n=5) which tends to be higher during earlier stage of symptomatic disease. Pearson correlation, r=−0.30, p=0.027. MSD signal is normalized to the lowest averaged reading of the sample duplicates. All samples were assayed on one plate. FIG. 10D is a graph illustrating ALS staging model based on the dynamic of neurofilament subunit and cryptic HDGFL2 accumulation in CSF of ALS patients. FIG. 10E is a graph illustrating ratios of pNfH/cryptic HDGFL2 and NfL/cryptic HDGFL2 in CSF of presymptomatic and symptomatic C9ORF72 mutation carriers. Using the ratio of pNfH and NfL to cryptic HDGFL2 in CSF, it may be possible to establish an ALS staging scale spanning from pre-symptomatic phase (ratio <1) to symptomatic phase (ratio >1).

FIG. 11 is a graph illustrating the rate of disease progression which does not correlate with CSF cryptic HDGFL2 measured by MSD assay in C9ORF72 mutation carriers.

FIG. 12 is a graph illustrating the reproducibility of cryptic HDGFL2 MSD assay. Nineteen C9ORF72 mutation carrier CSF samples were assayed on two different days. One set of samples had not been previously thawed, while the other set had been thawed and re-frozen one time. Normalized MSD signals were slightly decreased for the previously thawed samples, but relative signal levels were preserved (r=0.93; p<0.0001).

FIG. 13 is a graph illustrating cryptic HDGFL2 detection by MSD assay in CSF of both pre-symptomatic and symptomatic C9orf72 mutation carriers. Dotted line is drawn at the level under which control CSF signals typically fall when tested under the same assay parameters. MSD signal is normalized to the lowest averaged reading of the sample duplicates. All samples were assayed on one plate.

FIGS. 14A-14B are graphs illustrating cryptic HDGFL2 levels which tend to decrease across symptomatic disease progression. FIG. 14A shows MSD detection of cryptic HDGFL2 in longitudinal samples from 17 symptomatic C9orf72 mutation carriers at two or three time points in disease. The two time points for one individual correspond to a diagnosis of mild cognitive impairment at the first time point and FTD at the second. Another individual converted from ALS to ALS-FTD. All others had diagnoses of ALS (n=10), ALS-FTD (n=4), or FTD (n=1) at each time point. FIG. 14B shows the rate of change in cryptic HDGFL2 levels in the same individuals as in FIG. 14A. Cryptic HDGFL2 decreased over time in 82.35% (14/17) of the symptomatic individuals.

FIG. 15 is a graph illustrating the ratios of pNFH/cryptic HDGFL2 in CSF of pre-symptomatic and symptomatic C9orf72 mutation carriers.

FIG. 16 is a graph illustrating pNFH/cryptic HDGFL2 ratio which tends to increase across symptomatic disease progression.

FIG. 17 is a graph illustrating cryptic HDGFL2 detection by MSD assay which is higher in CSF of sporadic ALS patients (n=10) compared to healthy controls (n=5). MSD signal is normalized to the lowest averaged reading of the sample duplicates.

FIG. 18 shows photographs illustrating the localization of cryptic HDGFL2 in neurons of ALS-FTD brain. FTLD-TDP: frontotemporal lobar degeneration with TDP-43 pathology.

FIGS. 19A-19F illustrate the development of an MSD assay specific for cryptic HDGFL2. FIG. 19A is a schematic representation of a sandwich ELISA using Meso Scale Discovery (MSD) system. FIG. 19B shows photographs of immunoblots illustrating a band corresponding to WT HDGFL2 detectable by a goat antibody against native HDGFL2 (gTEA1.2) in lysates of non-transfected HeLa (ctrl), HeLa transfected to overexpress WT HDGFL2 (WT prot.), and HeLa transfected to overexpress cryptic HDGFL2 (cryptic prot.). FIG. 19C shows photographs of immunoblots illustrating a double band of the expected size in HeLa lysate co-transfected to overexpress both cryptic and WT HDGFL2 (cryptic+WT prot.) when probed with an antibody against WT HDGFL2. FIG. 19D is a graph illustrating a dose-dependent increase of MSD signal in lysate of HeLa overexpressing cryptic HDGFL2 compared to control HeLa lysate overexpressing WT HDGFL2. FIG. 19E is a graph illustrating elevated MSD signal of cryptic HDGFL2 overexpression lysate is specific to intact capture and detection antibodies. FIG. 19F is a graph illustrating lysates of HeLa cells transfected to overexpress the cryptic HDGFL2 protein were measured in a concentration series on the three MSD plates used for the CSF assays in this study, and these standard curves were analyzed by a four-parameter logistic (4PL) fit to normalize signal between plates.

FIGS. 20A-20D show standard curves used for quantification of cryptic HDGFL2. FIG. 20A shows photographs of immunoblots illustrating cryptic and WT HDGFL2 proteins purified. FIG. 20B is a graph illustrating a dose-dependent increase in MSD signal of purified cryptic HDGFL2 compared to WT HDGFL2. FIG. 20C is a graph illustrating that at lower concentrations, MSD signal for purified cryptic HDGFL2 falls in a linear range. FIG. 20D is a graph illustrating five standard curves from different MSD plates are shown and fitted with a four-parameter logistic (4PL) curve (solid line) used for signal normalization. Dotted lines represent the 95% confidence interval.

FIG. 21 is a graph illustrating cryptic HDGFL2 levels measured by MSD in C9ORF72-mutation carriers from NIH cohort.

FIG. 22 is a graph illustrating cryptic HDGFL2 levels measured by MSD in C9ORF72-mutation carriers and healthy controls from DIALS cohort.

FIG. 23 is a graph illustrating the effect of freeze-thaw on cryptic HDGFL2 MSD assay.

FIGS. 24A-24C show cryptic HDGFL2 detected by MSD assay in CSF of both presymptomatic and symptomatic C9ORF72-mutation carriers as well as sporadic ALS. FIG. 24A is a graph illustrating cryptic HDGFL2 levels (ng/ml) measured in CSF of healthy controls (n=16), presymptomatic (n=83) and symptomatic (n=66) C9ORF72-mutations carriers, and sporadic ALS (n=14). FIG. 24B is a graph illustrating change in cryptic HDGFL2 levels (ng/mL) in presymptomatic C9ORF72-mutations carriers across age. FIG. 24C is a graph illustrating change in cryptic HDGFL2 levels (ng/ml) in symptomatic C9ORF72-mutations carriers across disease progression.

### DETAILED DESCRIPTION OF THE INVENTION

The present invention is based on the seminal discovery that TDP-43 loss if function results in the loss of non-conserved cryptic exon splicing which generates neoepitopes, and more specifically to the development of antibodies and binding fragments thereof that specifically bind to said neoepitopes.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the invention provides a method of detecting TDP-43 loss of function in a subject including contacting a sample from the subject with an antibody or binding fragment thereof which specifically binds to a cryptic exon-encoded neoepitope, wherein the qcryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2), thereby detecting TDP-43 loss of function in the subject.

TAR DNA-binding protein 43 (TDP-43), or transactive response DNA binding protein 43 kDa, is a protein that in humans is encoded by the TARDBP gene. TDP-43 is 414 amino acid residues long that consists of 4 domains: an N-terminal domain spanning residues 1-76 (NTD) with a well-defined fold that has been shown to form a dimer or oligomer; 2 highly conserved folded RNA recognition motifs spanning residues 106-176 (RRM1) and 191-259 (RRM2), respectively, required to bind target RNA and DNA; an unstructured C-terminal domain encompassing residues 274-414 (CTD), which contains a glycine-rich region, is involved in protein-protein interactions, and harbors most of the mutations associated with familial amyotrophic lateral sclerosis. The NTD located between residues 1 and 76 is involved in TDP-43 polymerization. Indeed, dimers are formed by head-to-head interactions between NTDs, and the polymer thus obtained allows for pre-mRNA splicing. However, further oligomerization brings to more toxic accumulates. TDP-43 can aggregate with one another, accumulate, and spread using prion mechanisms of action. TDP-43 polypeptide or aggregates (or TDP-43 prions) are pathological and can be detected in subjects diagnosed with neurodegenerative diseases, associated with the accumulation of pathological protein in neurons, responsible for neurodegeneration.

TDP-43 is a transcriptional repressor that binds to chromosomally integrated TAR DNA and represses HIV-1 transcription. In addition, this protein regulates alternate splicing of the CFTR gene. TDP-43 has been shown to bind both DNA and RNA and have multiple functions in transcriptional repression, pre-mRNA splicing and translational regulation. Transcriptome-wide binding sites characterization revealed that thousands of RNAs are bound by TDP-43 in neurons. TDP-43 was originally identified as a transcriptional repressor that binds to chromosomally integrated trans-activation response element (TAR) DNA and represses HIV-1 transcription. It was also reported to regulate alternate splicing of the CFTR gene and the apoA-II gene. In spinal motor neurons TDP-43 has also been shown in humans to be a low molecular weight neurofilament (hNFL) mRNA-binding protein. It has also shown to be a neuronal activity response factor in the dendrites of hippocampal neurons suggesting possible roles in regulating mRNA stability, transport and local translation in neurons.

TDP-43 protein is a key element of the non-homologous end joining (NHEJ) enzymatic pathway that repairs DNA double-strand breaks (DSBs) in pluripotent stem cell-derived motor neurons. TDP-43 is rapidly recruited to DSBs where it acts as a scaffold for the further recruitment of the XRCC4-DNA ligase protein complex that then acts to seal the DNA breaks. In TDP-43 depleted human neural stem cell-derived motor neurons, as well as in sporadic ALS patients' spinal cord specimens there is significant DSB accumulation and reduced levels of NHEJ.

As used herein, a “TDP-43 loss of function” refers to any genetic or epigenetic modification that may result in a loss of one or more of TDP-43 function, including TDP-43 exon-splicing function. Many genetic or epigenetic alterations can result loss of function, nonlimiting examples include genetic mutations in the TARDBP gene (somatic or inherited mutations).

TARDBP is involved in the splicing of cryptic exons of selected mRNAs. As used herein, the term “cryptic exon” refers to splicing variants that may introduce frameshifts, exon fusions or stop codons, among other changes in the resulting mRNA. These mRNA anomalies can be reflected in translation of the corresponding proteins and result in the generation of “neoepitope”. The term “epitope” refers to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. As used herein a “neoepitope” refers to an epitope that does not normally exist, but that is newly created as the result of an alternative exon splicing, due to a TDP-43 loss of function.

The methods described herein are directed to the detection of a TDP-43 loss of function, which refers to the detection of a cryptic exon-encoded neoepitope, that results from the loss of function of TDP-43.

Cryptic exon-encoded neoepitope can be detecting using the antibodies described herein, and any method known in the art for antibody-based protein detection. Antibody-based protein detection methods include, but are not limited to immunohistochemistry, immunoblotting, immunofluorescence, and flow cytometry utilizing the antibodies described herein. Antibody binding is detected by techniques known in the art (e.g., radioimmunoassay, ELISA (enzyme-linked immunosorbent assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In some aspects, antibody binding is detected by detecting a label on the primary antibody. In another aspect, the primary antibody is detected by detecting binding of a secondary antibody or reagent to the primary antibody. In a further aspect, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized. In other embodiments, the immunoassay described in U.S. Pat. Nos. 5,599,677 and 5,672,480, each of which is herein incorporated by reference, can be used.

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject can visit a medical center to have the sample obtained and sent to the profiling center, or subjects can collect the sample themselves and directly send it to a profiling center. Where the sample comprises previously determined biological information, the information can be directly sent to the profiling service by the subject (e.g., an information card containing the information can be scanned by a computer and the data transmitted to a computer of the profiling center using an electronic communication system). Once received by the profiling service, the sample is processed and a profile is produced (e.g., expression data), specific for the diagnostic or prognostic information desired for the subject.

In one aspect, detecting TDP-43 loss of function includes detecting cryptic exon-encoded neoepitope in the sample from the subject.

A “sample” or “test sample” can be collected from a subject, in which the presence of, or the titer of cryptic exon-encoded neoepitope is sought to be measured. A “test sample” is a sample for which the presence (or absence) of or the cryptic exon-encoded neoepitope is sought to be analyzed. As used herein, a “sample” or “biological sample” is meant to refer to any “biological specimen” collected from a subject, and that is representative of the content or composition of the source of the sample, considered in its entirety. A sample can be collected and processed directly for analysis or be stored under proper storage conditions to maintain sample quality until analyses are completed. Ideally, a stored sample remains equivalent to a freshly collected specimen. The source of the sample can be an internal organ, vein, artery, or even a fluid. Non-limiting examples of sample include blood, plasma, urine, saliva, sweat, organ biopsy, cerebrospinal fluid (CSF), tear, semen, vaginal fluid, skin, and breast milk. In some aspects, the sample is a biological fluid. In various aspects, the biological fluid is selected from the group consisting of blood, cerebrospinal fluid (CSF), saliva, sputum, urine or another biofluid.

The term “subject” as used herein can refer to any individual or patient to which the methods described herein can be performed, and specifically from whom a sample can be collected. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

In another embodiment, the invention provides a method of detecting and/or diagnosing a TDP-43-associated disease in a subject including detecting TDP-43 loss of function in the subject, wherein detecting TDP-43 loss of function in the subject includes contacting a sample from the subject with an antibody or binding fragment thereof which specifically binds to a cryptic exon-encoded neoepitope, thereby detecting or diagnosing the TDP-43-associated disease in the subject.

As described above, the methods described herein allow for the detection of TDP-43 loss of function in the subject using the antibody of the present invention, by detecting cryptic exon-encoded neoepitope in a sample collected from the subject. TDP-43 loss of function is associated with the development of neurological and neurodegenerative diseases and disorders (also referred to as TDP-43-associated diseases. By detecting TDP-43 loss of function in a sample obtained from a subject, the methods described herein also allow for the detection of one of the symptoms of said TDP-43 diseases, which can be used to detect or diagnose the disease in the subject.

Various diseases are characterized by a TDP-43 loss of function. Non-limiting examples of TDP-43-associated disease include Alzheimer's disease (AD), amyotrophic lateral sclerosis (ALS), frontotemporal lobar degeneration (FTLD), inclusion body myositis (IBM), primary age-related tauopathy (PART)/Neurofibrillary tangle-predominant senile dementia, chronic traumatic encephalopathy (CTE), progressive supranuclear palsy (PSP), corticobasal degeneration (CBD), frontotemporal dementia and parkinsonism linked to chromosome 17 (FTDP-17), lytico-bodig disease (Parkinson-dementia complex of Guam), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, chronic traumatic encephalopathy, limbic-predominant age-related TDP-43 encephalopathy (LATE), multiple sclerosis (MS) and TDP-43 encephalopathy.

In some aspects, the TDP-43-associated disease is selected from the group consisting of AD, ALS, FTLD, IBM, CTE and MS.

Alzheimer's disease (AD) is a neurodegenerative disease that usually starts slowly and progressively worsens. It is the cause of 60-70% of cases of dementia. The most common early symptom is difficulty in remembering recent events. As the disease advances, symptoms can include problems with language, disorientation (including easily getting lost), mood swings, loss of motivation, self-neglect, and behavioral issues. Gradually, bodily functions are lost, ultimately leading to death. Although the speed of progression can vary, the typical life expectancy following diagnosis is three to nine years.

The disease process is largely associated with amyloid plaques, neurofibrillary tangles, and loss of neuronal connections in the brain. A probable diagnosis is based on the history of the illness and cognitive testing with medical imaging and blood tests to rule out other possible causes. Initial symptoms are often mistaken for normal aging. Examination of brain tissue is needed for a definite diagnosis, but this can only take place after death.

Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Degeneration is also present in brainstem nuclei particularly the locus coeruleus in the pons. Studies using MRI and PET have documented reductions in the size of specific brain regions in people with Alzheimer's disease as they progressed from mild cognitive impairment to Alzheimer's disease, and in comparison, with similar images from healthy older adults. Both Aβ plaques and neurofibrillary tangles are clearly visible by microscopy in brains of those with Alzheimer's disease, especially in the hippocampus. However, Alzheimer's disease may occur without neurofibrillary tangles in the neocortex. Plaques are dense, mostly insoluble deposits of beta-amyloid peptide and cellular material outside and around neurons. Tangles (neurofibrillary tangles) are aggregates of the microtubule-associated protein tau which has become hyperphosphorylated and accumulate inside the cells themselves. Although many older individuals develop some plaques and tangles as a consequence of aging, the brains of people with Alzheimer's disease have a greater number of them in specific brain regions such as the temporal lobe. Lewy bodies are not rare in the brains of people with Alzheimer's disease.

Amyotrophic lateral sclerosis (ALS), also known as motor neuron disease (MND) or Lou Gehrig's disease, is a neurodegenerative disease that results in the progressive loss of motor neurons that control voluntary muscles. ALS is the most common type of motor neuron disease. Early symptoms of ALS include stiff muscles, muscle twitches, and gradual increasing weakness and muscle wasting. Limb-onset ALS begins with weakness in the arms or legs, while bulbar-onset ALS begins with difficulty speaking or swallowing. Half of the people with ALS develop at least mild difficulties with thinking and behavior, and about 15% develop frontotemporal dementia. Most people experience pain. The affected muscles are responsible for chewing food, speaking, and walking. Motor neuron loss continues until the ability to eat, speak, move, and finally the ability to breathe is lost. ALS eventually causes paralysis and early death, usually from respiratory failure.

Most cases of ALS (about 90% to 95%) have no known cause and are known as sporadic ALS. However, both genetic and environmental factors are believed to be involved. The remaining 5% to 10% of cases have a genetic cause linked to a history of the disease in the family, and these are known as familial ALS. About half of these genetic cases are due to one of two specific genes. ALS and frontotemporal dementia (FTD) are considered to be part of a common disease spectrum (ALS-FTD) because of genetic, clinical, and pathological similarities. The underlying mechanism involves damage to both upper and lower motor neurons; in ALS-FTD, neurons in the frontal and temporal lobes of the brain die as well. The diagnosis is based on a person's signs and symptoms, with testing done to rule out other potential causes.

The defining feature of ALS is the death of both upper motor neurons (located in the motor cortex of the brain) and lower motor neurons (located in the brainstem and spinal cord). In ALS with frontotemporal dementia, neurons throughout the frontal and temporal lobes of the brain die as well. The pathological hallmark of ALS is the presence of inclusion bodies (abnormal aggregations of protein) known as Bunina bodies in the cytoplasm of motor neurons. In about 97% of people with ALS, the main component of the inclusion bodies is TDP-43 protein; however, in those with SOD1 or FUS mutations, the main component of the inclusion bodies is SOD1 protein or FUS protein, respectively. The gross pathology of ALS, which are features of the disease that can be seen with the naked eye, include skeletal muscle atrophy, motor cortex atrophy, sclerosis of the corticospinal and corticobulbar tracts, thinning of the hypoglossal nerves (which control the tongue), and thinning of the anterior roots of the spinal cord. Aside from the death of motor neurons, two other characteristics common to most ALS variants are focal initial pathology, meaning that symptoms start in a single spinal cord region, and progressive continuous spread, meaning that symptoms spread to additional regions over time. Prion-like propagation of misfolded proteins from cell to cell may explain why ALS starts in one area and spreads to others. The glymphatic system may also be involved in the pathogenesis of ALS.

Frontotemporal dementia (FTD), or frontotemporal degeneration disease, or frontotemporal neurocognitive disorder, encompasses several types of dementia involving the progressive degeneration of frontal and temporal lobes. FTDs broadly present as behavioral or language disorders with gradual onsets. The three main subtypes or variant syndromes are a behavioral variant (bvFTD) previously known as Pick's disease, and two variants of primary progressive aphasia—semantic variant (svPPA), and nonfluent variant (nfvPPA). Two rare distinct subtypes of FTD are neuronal intermediate filament inclusion disease (NIFID), and basophilic inclusion body disease. Other related disorders include corticobasal syndrome and FTD with amyotrophic lateral sclerosis (ALS) FTD-ALS also called FTD-MND.

Frontotemporal dementias are mostly early-onset syndromes that are linked to frontotemporal lobar degeneration (FTLD), which is characterized by progressive neuronal loss predominantly involving the frontal or temporal lobes, and a typical loss of more than 70% of spindle neurons, while other neuron types remain intact.

There are three main histological subtypes found at post-mortem: FTLD-tau, FTLD-TDP, and FTLD-FUS. In rare cases, patients with clinical FTD were found to have changes consistent with Alzheimer's disease on autopsy. The most severe brain atrophy appears to be associated with behavioral variant FTD, and corticobasal degeneration. With regard to the genetic defects that have been found, repeat expansion in the C9orf72 gene is considered a major contribution to frontotemporal lobar degeneration, although defects in the GRN and MAPT genes are also associated with it.

Inclusion body myositis (IBM) (sometimes called sporadic inclusion body myositis, sIBM) is the most common inflammatory muscle disease in older adults. The disease is characterized by slowly progressive weakness and wasting of both proximal muscles (located on or close to the torso) and distal muscles (close to hands or feet), most apparent in the finger flexors and knee extensors. In IBM, two processes appear to occur in the muscles in parallel, one autoimmune and the other degenerative. Inflammation is evident from the invasion of muscle fibers by immune cells. Degeneration is characterized by the appearance of holes, deposits of abnormal proteins, and filamentous inclusions in the muscle fibers. sIBM is a rare disease, with a prevalence ranging from 1 to 71 individuals per million. Weakness comes on slowly (over months to years) in an asymmetric manner and progresses steadily, leading to severe weakness and wasting of arm and leg muscles.

Chronic traumatic encephalopathy (CTE) is a neurodegenerative disease linked to repeated trauma to the head. The encephalopathy symptoms can include behavioral problems, mood problems, and problems with thinking. The disease often gets worse over time and can result in dementia. It is unclear if the risk of suicide is altered. Most documented cases have occurred in athletes involved in striking-based combat sports, such as boxing, kickboxing, mixed martial arts, and Muay Thai—hence its original name dementia pugilistica (Latin for “fistfighter's dementia”)—and contact sports such as American football, Australian rules football, professional wrestling, ice hockey, rugby, and association football (soccer), in semi-contact sports such as baseball and basketball, and military combat arms occupations. There is no specific treatment for the disease. Rates of CTE have been found to be about 30% among those with a history of multiple head injuries; however, population rates are unclear. Research in brain damage as a result of repeated head injuries began in the 1920s, at which time the condition was known as dementia pugilistica or “fistfighter's dementia”, “boxer's madness”, or “punch drunk syndrome”. It has been proposed that the rules of some sports be changed as a means of prevention.

The neuropathological appearance of CTE is distinguished from other tauopathies, such as Alzheimer's disease. The four clinical stages of observable CTE disability have been correlated with tau pathology in brain tissue, ranging in severity from focal perivascular epicenters of neurofibrillary tangles in the frontal neocortex to severe tauopathy affecting widespread brain regions. The primary physical manifestations of CTE include a reduction in brain weight, associated with atrophy of the frontal and temporal cortices and medial temporal lobe. The lateral ventricles and the third ventricle are often enlarged, with rare instances of dilation of the fourth ventricle. Other physical manifestations of CTE include anterior cavum septi pellucidi and posterior fenestrations, pallor of the substantia nigra and locus ceruleus, and atrophy of the olfactory bulbs, thalamus, mammillary bodies, brainstem and cerebellum. As CTE progresses, there may be marked atrophy of the hippocampus, entorhinal cortex, and amygdala.

Multiple sclerosis (MS), also known as encephalomyelitis disseminata, is the most common demyelinating disease, in which the insulating covers of nerve cells in the brain and spinal cord are damaged. This damage disrupts the ability of parts of the nervous system to transmit signals, resulting in a range of signs and symptoms, including physical, mental, and sometimes psychiatric problems. Specific symptoms can include double vision, blindness in one eye, muscle weakness, and trouble with sensation or coordination. MS takes several forms, with new symptoms either occurring in isolated attacks (relapsing forms) or building up over time (progressive forms). Between attacks, symptoms may disappear completely, although permanent neurological problems often remain, especially as the disease advances. While the cause is unclear, the underlying mechanism is thought to be either destruction by the immune system or failure of the myelin-producing cells. Proposed causes for this include genetics and environmental factors, such as viral infections. MS is usually diagnosed based on the presenting signs and symptoms and the results of supporting medical tests.

Multiple sclerosis is the most common immune-mediated disorder affecting the central nervous system. In 2015, about 2.3 million people were affected globally, with rates varying widely in different regions and among different populations. In that year, about 18,900 people died from MS, up from 12,000 in 1990. The disease usually begins between the ages of twenty and fifty and is twice as common in women as in men. MS was first described in 1868 by French neurologist Jean-Martin Charcot. The name multiple sclerosis refers to the numerous glial scars (or sclerae—essentially plaques or lesions) that develop on the white matter of the brain and spinal cord. As of 2009 a number of new treatments and diagnostic methods are under development.

The three main characteristics of MS are the formation of lesions in the central nervous system (also called plaques), inflammation and the destruction of myelin sheaths of neurons. These features interact in a complex and not yet fully understood manner to produce the breakdown of nerve tissue and in turn the signs and symptoms of the disease. Cholesterol crystals are believed both to impair myelin repair and to aggravate inflammation. MS is believed to be an immune-mediated disorder that develops from an interaction of the individual's genetics and as yet unidentified environmental causes. Damage is believed to be caused, at least in part, by attack on the nervous system by a person's own immune system.

In some aspects, the TDP-43 associated disease is an early stage of the disease or in a pre-symptomatic phase of the disease.

The phrases “early stage” and “pre-symptomatic phase”, as used herein, are meant to refers to any time in the life course of a disease during which the subject affected by said disease does not present or experience-yet-any symptoms of the disease. Or experience minimal symptoms of the disease (e.g., symptoms that do not impact on the subject's life). For example, during the pre-symptomatic phase of a disease, a subject can be asymptomatic, or present symptoms that are usually not sufficient by themselves to ascertain of development of a disease (e.g., the subject experience symptoms that are not specific to the disease).

The present invention is based on the discovery of genes whose mRNA are normally protected from splicing errors by TDP-43, and for whom a TDP-43 loss of function results in the generation of neoepitope. In one aspect, the cryptic exon-encoded neoepitope is within (i) HDGFL2 (HDGF Like 2), (ii) ACTL6B (Actin Like 6B), (iii) ARHGAP32 (Rho GTPase Activating Protein 32), (iv) EPB41L4A (Erythrocyte Membrane Protein Band 4.1 Like 4A), (v) SLC24A3 (Solute Carrier Family 24 Member 3), (vi) CDO1 (Cysteine Dioxygenase Type 1), (vii) AGRN (Agrin), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2).

HDGFL2, or hepatoma-derived growth factor-related protein 2 is a protein that acts as an epigenetic regulator of myogenesis in cooperation with DPF3a (isoform 2 of DPF3/BAF45C). It associates with the BAF complex via its interaction with DPF3a and HDGFL2-DPF3a activates myogenic genes by increasing chromatin accessibility through recruitment of SMARCA4/BRG1/BAF190A (ATPase subunit of the BAF complex) to myogenic gene promoters. HDGFL2 promotes the repair of DNA double-strand breaks (DSBs) through the homologous recombination pathway by facilitating the recruitment of the DNA endonuclease RBBP8 to the DSBs. HDGFL2 preferentially binds to chromatin regions marked by H3K9me3, H3K27me3 and H3K36me2, and is involved in cellular growth control, through the regulation of cyclin D1 expression.

Actin-like protein 6B is a protein that in humans is encoded by the ACTL6B gene. The protein encoded by this gene is a member of a family of actin-related proteins (ARPs) which share significant amino acid sequence identity to conventional actins. Both actins and ARPs have an actin fold, which is an ATP-binding cleft, as a common feature. The ARPs are involved in diverse cellular processes, including vesicular transport, spindle orientation, nuclear migration and chromatin remodeling. This gene encodes a subunit of the BAF (BRG1/brm-associated factor) complex in mammals, which is functionally related to SWI/SNF complex in S. cerevisiae and Drosophila; the latter is thought to facilitate transcriptional activation of specific genes by antagonizing chromatin-mediated transcriptional repression. This subunit may be involved in the regulation of genes by structural modulation of their chromatin, specifically in the brain.

Rho GTPase-activating protein 32 is a protein that in humans is encoded by the RICS gene. RICS has two known isoforms, RICS that are expressed primarily at neurite growth cones, and at the post synaptic membranes, and PX-RICS which is more widely expressed in the endoplasmic reticulum, Golgi apparatus and endosomes. The only known domain of the RICS is the RhoGAP domain, whilst PX-RICS has an additional Phox homology and SH3 domain. RICS (a.k.a. GRIT/Arhgap32) is a neuron-associated GTPase-activating protein that may regulate dendritic spine morphology and strength by modulating Rho GTPase activity.

Agrin is a large proteoglycan whose best-characterized role is in the development of the neuromuscular junction during embryogenesis. Agrin is named based on its involvement in the aggregation of acetylcholine receptors during synaptogenesis. In humans, this protein is encoded by the AGRN gene. Agrin has nine domains homologous to protease inhibitors. It may also have functions in other tissues and during other stages of development. It is a major proteoglycan component in the glomerular basement membrane and may play a role in the renal filtration and cell-matrix interactions.

The IgLON proteins are a family of five cell-adhesion molecules IgLON 1, 2, 3, 4 & 5, which assist in neuronal growth and connections among nerve cells and help in brain evolution and maturation to maintain integrity of the blood brain barrier. Abnormal pTau deposits seen in several brains, brain stems and upper cervical cords shown by neuro-immuno-histochemistry studies of brain tissue from these regions without inflammatory cells differentiate this entity from other autoimmune encephalitis. IgLON5 refers to a cell surface protein involved in promoting connections among nerve cells. Prevalence of the HLA-DRB1*10:01 allele was greatly increased in people with anti-IgLON5 disease. The sleep problems seen in this disorder are insomnia, sleep related abnormal movements called parasomnias which may be seen in both REM and NREM sleep and poor efficiency of sleep. Respiratory problems related to sleep disorder such as obstructive sleep apnea (OSA) and jerky stertorous breathing were noted in more than half the cases. Anti-IgLON5 disease is a neurodegenerative autoimmune disease. It is characterized by parasomnias and chorea—an involuntary movement disorder.

Dynamin-1 (DNM1) is a protein that in humans is encoded by the DNM1 gene. This gene encodes a member of the dynamin subfamily of GTP-binding proteins. The encoded protein possesses unique mechanochemical properties used to tubulate and sever membranes and is involved in clathrin-mediated endocytosis and other vesicular trafficking processes. Actin and other cytoskeletal proteins act as binding partners for the encoded protein, which can also self-assemble leading to stimulation of GTPase activity. More than sixty highly conserved copies of the 3′ region of this gene are found elsewhere in the genome, particularly on chromosomes Y and 15. Alternatively spliced transcript variants encoding different isoforms have been described.

In enzymology, an alanine—tRNA ligase or transferase is an enzyme that catalyzes the chemical reaction:

ATP+L-alanine+tRNAAla=\rightleftharpoons AMP+diphosphate+L-alanyl-tRNAAla

The 3 substrates of this enzyme are ATP, L-alanine, and tRNA (Ala), whereas its 3 products are AMP, diphosphate, and L-alanyl-tRNA (Ala). This enzyme belongs to the family of ligases, to be specific those forming carbon-oxygen bonds in aminoacyl-tRNA and related compounds. The systematic name of this enzyme class is L-alanine: RNAAla ligase (AMP-forming). Other names in common use include alanyl-tRNA synthetase, alanyl-transfer ribonucleate synthetase, alanyl-transfer RNA synthetase, alanyl-transfer ribonucleic acid synthetase, alanine-transfer RNA ligase, alanine transfer RNA synthetase, alanine tRNA synthetase, alanine translase, alanyl-transfer ribonucleate synthase, AlaRS, and Ala-tRNA synthetase. This enzyme participates in alanine and aspartate metabolism and aminoacyl-trna biosynthesis.

Peroxidasin (PXDN) is a protein that in humans is encoded by the PXDN gene. Peroxidasin requires ionic bromine as a co-factor, making bromine an essential element for human life. Mutations in PXDN are associated with microphthalmia.

N-terminal EF-hand calcium-binding protein 2 (NECAB2) is a protein that in humans is encoded by the NECAB2 gene. Model organisms have been used in the study of NECAB2 function. A conditional knockout mouse line, called Necab2tm1a (KOMP) Wtsi was generated as part of the International Knockout Mouse Consortium program—a high-throughput mutagenesis project to generate and distribute animal models of disease to interested scientists—at the Wellcome Trust Sanger Institute. Male and female animals underwent a standardized phenotypic screen to determine the effects of deletion. Twenty-five tests were carried out on mutant mice but no significant abnormalities were observed.

In one aspect, wherein the cryptic exon-encoded neoepitope is within HDGFL2.

In another aspect, the method further includes detecting one or more additional TDP-43-associated biomarkers in the sample.

By “TDP-43-associated biomarkers” it is meant any biomarkers other than HDGFL2, ACTL6B, ARHGAP32, EPB41L4A, SLC24A3, CDO1, AGRN, IGLON5, DNM1, AARS1, PXDN, and NECAB2, that is specifically differentially expressed in subject suffering from a TDP-43 associated disease. A biomarker can either be overexpressed (e.g., the expression is higher in subject having the disease that in subject not having the disease), expressed (e.g., the expression is present in subject having the disease but absent in subject not having the disease), decreased (e.g., the expression is lower in subject having the disease that in subject not having the disease) or repressed (e.g., the expression is absent in subject having the disease but present in subject not having the disease) in subject having a TDP-43 associated disease as compared to subject not having such a disease.

In some aspects, the one or more TDP-43-associated biomarkers are selected from the group consisting of neurofilament (NF), tau, amyloid-β, α-synuclein, and combinations thereof.

Neurofilament light polypeptide (NfL) or simply neurofilament (NF), also known as neurofilament light chain, is a neurofilament protein that in humans is encoded by the NEFL gene. Neurofilament light chain is a biomarker that can be measured with immunoassays in cerebrospinal fluid and plasma and reflects axonal damage in a wide variety of neurological disorders. It is a useful marker for disease monitoring in amyotrophic lateral sclerosis, multiple sclerosis, Alzheimer's disease, and more recently Huntington's disease. Higher numbers have been associated with increased mortality. It is associated with Charcot-Marie-Tooth disease 1F and 2E.

Tau is a natively unstructured protein expressed as 6 isoforms in the adult human brain that result from alternative splicing of the MAPT gene. Tau is mainly known for its ability to stabilize microtubules within axons of neurons. Tau isoforms are composed of either 3 or 4 microtubule-binding repeats (MTBRs; 3R or 4R), which mediate binding of tau to microtubules. Aberrant misfolding of tau leads to fibrillization and the formation of paired helical filaments with all 6 tau isoforms that constitute neurofibrillary tangles (NFTs). Misfolded tau, or “tau seeds” are capable of initiating aggregation of various forms of tau. As used herein, the term “tau seed” refers to a tau aggregate—or seed—that is a misfolded tau protein or fragment thereof, capable of recruiting normal, soluble tau into a fibrillar conformation. Tau seeds can spread, transmitting the aggregated tau from cell to cell via prion-like mechanisms. Upon uptake and processing, the misfolded seed tau seed can initiate templated fibrilization and recruit native tau monomer by direct protein-protein interactions between a pathological tau seed and naive cellular tau, to form new pathologic fibril in the recipient cell. The conversion of a protein from a monomer to a large, ordered multimer can occur by several mechanisms, but the first step likely involves the formation of a seed. A seed is potentially transitory, arising from an equilibrium between two states: one relatively aggregation-resistant, and another that is short-lived. A seed could be a single molecule, or several. Based on extrapolation from kinetic aggregation studies, it is likely that a critical seed for tau and polyglutamine peptide amyloid formation is a single molecule or a tau multimer. Therefore, the term “seed” is used to refer to the structure that serves as a template for homotypic fibril growth and can range in size from a protein monomer to a multimeric assembly. For example, a seed can refer to any misfolded protein capable of initiating aggregation of various forms of tau, and can therefore comprise 1, 2, 3, 4, 5, 10, 20, 30, 40, 50 or more monomers or 50, 40, 30, 20, 10, 5, 4, 3, 2 or less monomers. As used herein a “tau repeat domain” refers to a domain or portion of a tau protein or fragment capable of forming self-replicating assemblies (e.g., tau protein or aggregates thereof capable of inducing protein-protein interaction and therefore further tau aggregates, and capable of transmission from one cell to the other). A “tau repeat domain” corresponds to a portion of a tau protein that can interact with another tau protein to form protein-protein interactions, and thus generate fibrils. A Tau repeat domain comprises three or four 31-32-residue imperfect repeats that form the core of tau filaments and is capable of self-assembling into filaments in vitro. Therefore, a “tau repeat domain” can be used to refer to a microtubule-binding repeat domain of a tau protein described herein.

Amyloid beta (Aβ or Abeta) are peptides of 36-43 amino acids that are the main component of the amyloid plaques found in the brains of people with Alzheimer's disease. The peptides derive from the amyloid precursor protein (APP), which is cleaved by beta secretase and gamma secretase to yield Aβ in a cholesterol-dependent process and substrate presentation. Aβ molecules can aggregate to form flexible soluble oligomers which may exist in several forms. It is now believed that certain misfolded oligomers (known as “seeds”) can induce other Aβ molecules to also take the misfolded oligomeric form, leading to a chain reaction akin to a prion infection. The oligomers are toxic to nerve cells. The other protein implicated in Alzheimer's disease, tau protein, also forms such prion-like misfolded oligomers, and there is some evidence that misfolded Aβ can induce tau to misfold. Aβ is the main component of amyloid plaques, extracellular deposits found in the brains of people with Alzheimer's disease. Aβ can also form the deposits that line cerebral blood vessels in cerebral amyloid angiopathy. The plaques are composed of a tangle of Aß oligomers and regularly ordered aggregates called amyloid fibrils, a protein fold shared by other peptides such as the prions associated with protein misfolding diseases.

Alpha-synuclein (α-syn) is a protein that, in humans, is encoded by the SNCA gene. α-syn is abundant in the brain (predominantly expressed in the neocortex, hippocampus, substantia nigra, thalamus, and cerebellum), and mainly expressed at presynaptic terminals of neurons where it interacts with phospholipids and proteins. At least three isoforms of synuclein are produced through alternative splicing, but the mainly expressed form of the protein is the full-length protein of 140 amino acids, which includes three distinct domains. Residues 1-60 encode an amphipathic N-terminal region dominated by four 11-residue repeats including the consensus sequence KTKEGV having a structural alpha helix propensity similar to apolipoproteins-binding domains. It is a highly conserved terminal that interacts with acidic lipid membranes, and all the discovered point mutations of the SNCA gene are located within this terminal. Residues 61-95 encode a central hydrophobic region which includes the non-amyloid-β component (NAC) region, involved in protein aggregation. This domain is unique to alpha-synuclein among the synuclein family. Residues 96-140 encode a highly acidic and proline-rich region which has no distinct structural propensity. This domain plays an important role in the function, solubility and interaction of alpha-synuclein with other proteins. Unmutated α-synuclein forms a stably folded tetramer that resists aggregation, however, in pathological conditions, α-syn can aggregate and form insoluble fibrils. The aggregation mechanism of alpha-synuclein is uncertain and might rely on structured intermediate rich in beta structure that can be the precursor of aggregation and, ultimately, Lewy bodies. Unfolded monomer can aggregate first into small oligomeric species that can be stabilized by β-sheet-like interactions and then into higher molecular weight insoluble fibrils. Protein modifications such as phosphorylation (such as phosphorylation at Ser129 by polo-like kinase 2 (PLK2) kinase), truncation (through proteases such as calpains), and nitration (probably through nitric oxide (NO) or other reactive nitrogen species that are present during inflammation), modify synuclein such that it has a higher tendency to aggregate. The addition of ubiquitin to Lewy bodies is a secondary process to deposition.

Genetic alterations of the SNCA gene, can also result in aberrant polymerization of α-syn into insoluble fibrils, which are associated with several neurodegenerative diseases (synucleinopathies).

In an additional embodiment, the invention provides a method of monitoring a TDP-43-associated disease progression and/or response to a TDP-43-associated disease therapy in a subject including detecting cryptic exon-encoded neoepitopes in a sample from the subject, wherein detecting cryptic exon-encoded neoepitopes in the sample includes contacting the sample with an antibody or binding fragment thereof which specifically binds to a cryptic exon-encoded neoepitope, thereby monitoring the TDP-43-associated disease progression and/or response to therapy in the subject.

By “monitoring” it is meant that the methods described herein are used to study the evolution of the disease over time, to assess any changes in the parameters that evaluated by the described methods (e.g., assessing the evolution of the number and/or amount of cryptic exon-encoded neoepitopes detected in a sample obtained from a subject over time, for example in response to a treatment). Such monitoring is used to assess the progression or lack thereof of the disease and can be used for example to identify a time when a therapy should be initiated, stopped, discontinued, or changed.

In one aspect, the cryptic exon-encoded neoepitope is within (i) HDGFL2, (ii) ACTL6B, (iii) ARHGAP32, (iv) EPB41L4A, (v) SLC24A3, (vi) CDO1, (vii) AGRN, (viii) IGLON5, (ix) DNM1, (x) AARS1, (xi) PXDN, or (xii) NECAB2.

In another aspect, detecting is repeated over time. In some aspects, an increase in the detection of a cryptic exon-encoded neoepitope or an increase in a number of cryptic exon-encoded neoepitopes detected in the sample in a first detection as compared to a second detection is indicative of disease progression and/or of an absence of response to the therapy. In such a case, where progression of the disease is identified under a treatment with a given therapy, a physician may assert that the therapy needs to be stopped, and optionally changed for another therapy.

In other aspects, a decrease in the detection of a cryptic exon-encoded neoepitope or a decrease in a number of cryptic exon-encoded neoepitopes detected in the sample in a first detection as compared to a second detection is indicative of an absence of disease progression, a disease regression, and/or of a response to the therapy. Alternatively, when disease regression in identified under a treatment with a given therapy, a physician may assert that the therapy needs to be pursued to maintain the effects observed, or discontinued if no additional benefits are to be expected from the therapy.

In another aspect, the newly detection of cryptic exon-encoded neoepitope, when such detection was absent in a previous test is indicative of a development of the disease (e.g., the disease is diagnosed), which can coincide with the time for a physician to decide that a therapy should be initiated for the subject.

In a further embodiment, the invention provides a method of selecting a patient for enrollment in a clinical trial including detecting cryptic exon-encoded neoepitopes in a sample from the subject, wherein detecting cryptic exon-encoded neoepitopes in the sample includes contacting the sample with an antibody or binding fragment thereof which specifically binds to a cryptic exon-encoded neoepitope, thereby selecting the patient for enrollment in the clinical trial.

Clinical trials are meant to evaluate the efficacy and safety of new drugs products for a disease indication. The methods described herein allow for the detection of TDP-43 loss of function in a subject. Such loss of function can translate in a variety of changes in cells from a subject e.g., the newly expression of cryptic exon-encoded neoepitope within HDGFL2, ACTL6B, ARHGAP32, EPB41L4A, SLC24A3, CDO1, AGRN, IGLON5, DNM1, AARS1, PXDN, and/or NECAB2. Those neoepitope, at least, can be the target of drug drugs for the treatment of TDP-43-associated diseases. The detection of such cryptic neoepitopes is a means to select a patient having clearly defined TDP-43-associated disease (defined by the specific cryptic neoepitope expression or combination of specific cryptic neoepitopes), which allows for a tailored enrollment in a clinical trial that target said specific cryptic neoepitope or combination of specific cryptic neoepitopes.

In another aspect, the clinical trial is investigating a therapy for the treatment of a TDP-43 associated disease.

In one aspect, the cryptic exon-encoded neoepitope is within (i) HDGFL2, (ii) ACTL6B, (iii) ARHGAP32, (iv) EPB41L4A, (v) SLC24A3, (vi) CDO1, (vii) AGRN, (viii) IGLON5, (ix) DNM1, (x) AARS1, (xi) PXDN, or (xii) NECAB2.

In some aspects, the TDP-43 associated disease is characterized by the expression of a cryptic exon-encoded neoepitope.

In one embodiment, the invention provides a method of predicting pheno-conversion of a TDP-43 associated disease in a subject including determining a ratio of a TDP-43-associated biomarker to a cryptic exon-encoded neoepitope in a sample from the subject, thereby predicting pheno-conversion in the subject.

As used herein, the term “pheno-conversion” refers to a time in the life course of a disease where a subject affected by said disease switched from being asymptomatic (known to have the disease, for example by being aware of a mutation that is responsible for a disease, but without experiencing any symptoms of the disease) to symptomatic (known to have the disease and experiencing symptoms of the disease). Pheno-conversion is an important concept, as it is relevant to the timing when patient may start taking medications for their disease, or when they may become eligible to enroll into clinical trials. Depending on the knowledge and understanding of the disease, pheno-conversion can be identified by the evaluation of criterion that may not translate yet into symptoms that impact a subject's life, and therefore may have implication in maintaining a certain quality of life for a patient, if a medication can be started early on when pheno-conversion is detected for example.

In one aspect, the TDP-43-associated biomarker is selected from the group consisting of phosphorylated neurofilament heavy chain (pNFH), neurofilament light chain (NFL), tau, amyloid-β, α-synuclein, and combinations thereof.

In another aspect, determining a ratio of pNFH to a cryptic exon-encoded neoepitope includes: (i) determining a level of cryptic exon-encoded neoepitope in a sample from the subject, and (ii) determining a level of phosphorylated neurofilament heavy chain in the sample from the subject.

In one aspect, a ratio greater than 1 is indicative of a symptomatic stage of the TDP-43 associated disease. In another aspect, a ratio lesser than 1 is indicative of a pre-symptomatic stage of the TDP-43 associated disease. In another aspect, determining a level of cryptic exon-encoded neoepitope in a sample includes contacting the sample with an antibody or binding fragment thereof which specifically binds to a cryptic exon-encoded neoepitope.

In some aspects, the cryptic exon-encoded neoepitope is within (i) HDGFL2, (ii) ACTL6B, (iii) ARHGAP32, (iv) EPB41L4A, (v) SLC24A3, (vi) CDO1, (vii) AGRN, (viii) IGLON5, (ix) DNM1, (x) AARS1, (xi) PXDN, or (xii) NECAB2.

In various aspects, the cryptic exon-encoded neoepitope is HDGFL2.

In some aspects, the TDP-43 associated disease is amyotrophic lateral sclerosis (ALS).

In various aspects, the subject carries a C9ORF72 mutation.

In another embodiment, the invention provides a kit including a) one or more antibodies or binding fragment thereof which specifically bind to a cryptic exon-encoded neoepitopes; and b) instructions to use the antibodies of a) to detect TDP-43 loss of function in a sample, wherein the cryptic exon-encoded neoepitope is within (i) HDGFL2, (ii) ACTL6B, (iii) ARHGAP32, (iv) EPB41L4A, (v) SLC24A3, (vi) CDO1, (vii) AGRN, (viii) IGLON5, (ix) DNM1, (x) AARS1, (xi) PXDN, or (xii) NECAB2.

The present invention provides kits for the detection of a cryptic exon-encoded neoepitopes resulting from TDP-43 loss of function. In some embodiments, the kits contain antibodies specific for the cryptic neoepitopes, such as the antibodies of the present invention, in addition to detection reagents and buffers. In some embodiments, the kits contain all of the components necessary and/or sufficient to perform a detection assay, including all controls, directions for performing assays, and any necessary software for analysis and presentation of results.

In one aspect, the kit further includes an antibody or binding fragment thereof which specifically bind to phosphorylated neurofilament heavy chain (pNFH).

In an additional embodiment, the invention provides an antibody or binding fragment thereof which specifically binds to a cryptic exon-encoded neoepitope, wherein the cryptic exon-encoded neoepitope is an epitope resulting from a splicing incorporation of an exon normally repressed by TDP-43, and wherein the cryptic exon-encoded neoepitope is within (i) HDGFL2, (ii) ACTL6B, (iii) ARHGAP32, (iv) EPB41L4A, (v) SLC24A3, (vi) CDO1, (vii) AGRN, (viii) IGLON5, (ix) DNM1, (x) AARS1, (xi) PXDN, or (xii) NECAB2.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains respectively.

Experimentally, antibodies can be cleaved with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., Fundamental Immunology (Paul ed., 3d ed. 1993). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries. The term antibody also includes bivalent or bispecific molecules, diabodies, triabodies, and tetrabodies. Bivalent and bispecific molecules are known in the art.

The Fab fragment contains the constant domain of the light chain and the first constant domain (CH1) of the heavy chain. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F (ab′) 2 antibody fragments originally were produced as pairs of Fab′ fragments which have hinge cysteines between them. Other chemical couplings of antibody fragments are also known. The Fc region of an antibody is the tail region of an antibody that interacts with cell surface receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment is essential for Fc receptor-mediated activity. The N-glycans attached to this site are predominantly core-fucosylated diantennary structures of the complex type. In addition, small amounts of these N-glycans also bear bisecting GlcNAc and α-2,6 linked sialic acid residues. Fc-Fusion proteins (also known as Fc chimeric fusion protein, Fc-Ig, Ig-based Chimeric Fusion protein and Fc-tag protein) are composed of the Fc domain of IgG genetically linked to a peptide or protein of interest. Fc-Fusion proteins have become valuable reagents for in vivo and in vitro research. The Fc-fused binding partner can range from a single peptide, a ligand that activates upon binding with a cell surface receptor, signaling molecules, the extracellular domain of a receptor that is activated upon dimerization or as a bait protein that is used to identify binding partners in a protein microarray. One of the most valuable features of the Fc domain in vivo, is it can dramatically prolong the plasma half-life of the protein of interest, which for bio-therapeutic drugs, results in an improved therapeutic efficacy; an attribute that has made Fc-Fusion proteins attractive bio-therapeutic agents.

The Fc fusion protein may be part of a pharmaceutical composition including an Fc fusion protein and a pharmaceutically acceptable carrier excipients or carrier. Pharmaceutically acceptable carriers, excipients or stabilizers are well known in the art (Remington's Pharmaceutical Sciences, 16th edition, Osol, A. Ed. (1980)). Acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). “Fv” is the minimum antibody fragment which contains a complete antigen-recognition and-binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. “Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

References to “VH” or a “VH” refer to the variable region of an immunoglobulin heavy chain, including an Fv, scFv, a disulfilde-stabilized Fv (dsFv) or Fab. References to “VL” or a “VL” refer to the variable region of an immunoglobulin light chain, including of an Fv, scFv, dsFv or Fab.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. The numbering of the light and heavy chain variable regions described herein is in accordance with Kabat (see, e.g., Johnson et al., (2001) “Kabat Database and its applications: future directions” Nucleic Acids Research, 29:205-206; and the Kabat Database of Sequences of Proteins of Immunological Interest, Feb. 22, 2002 Dataset) unless otherwise indicated.

The positions of the CDRs and framework regions can be determined using various well-known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273 (4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29 (1): 207-9 (2001); MacCallum et al, Antibody-antigen interactions: Contact analysis and binding site topography, J Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

The term “specifically binds,” “binding specificity,” “specifically binds to an antibody” or “specifically immunoreactive with,” when referring to an epitope, refers to a binding reaction which is determinative of the presence of the epitope in a heterogeneous population of proteins and other biologics. Thus, under designated immunoassay conditions, the specified antibodies bind to a particular epitope at least two times the background and more typically more than 10 to 100 times background. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, ANTIBODIES, A LABORATORY MANUAL, Cold Spring Harbor Press, New York (1988) and Harlow & Lane, USING ANTIBODIES, A LABORATORY MANUAL, Cold Spring Harbor Press, New York (1999), for a description of immunoassay formats and conditions that can be used to determine specific immunoreactivity. As used herein, “specifically binds” means that an antibody binds to a protein with a Kd of at least about 0.1 mM, at least about 1 μM, at least about 0.1 μM or better, or 0.01 μM or better.

The antibodies described herein are proteins or polypeptides encoded by nucleic acid sequences. “Nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). As appreciate by one of skill in the art, the complement of a nucleic acid sequence can readily be determined from the sequence of the other strand. Thus, any particular nucleic acid sequence set forth herein also discloses the complementary strand.

“Polypeptide,” “peptide” and “protein” are used interchangeably herein to refer to a polymer of amino acid residues. The terms apply to naturally occurring amino acid polymers, as well as, amino acid polymers in which one or more amino acid residue is an artificial chemical mimetic of a corresponding naturally occurring amino acid. The amino acid sequences of the antibodies described herein can include naturally occurring and synthetic amino acids.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Conservatively modified variants” applies to both nucleic acid and amino acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. “Tertiary structure” refers to the complete three-dimensional structure of a polypeptide monomer. Domains are portions of a polypeptide that form a compact unit of the polypeptide and are typically 50 to 350 amino acids long. Typical domains are made up of sections of lesser organization such as stretches of β-sheet and α-helices. Quaternary structure” refers to the three-dimensional structure formed by the noncovalent association of independent tertiary units.

The terms “isolated” or “substantially purified,” when applied to a nucleic acid or protein, denotes that the nucleic acid or protein is essentially free of other cellular components with which it is associated in the natural state. It is preferably in a homogeneous state, although it can be in either a dry or aqueous solution. Purity and homogeneity are typically determined using analytical chemistry techniques such as polyacrylamide gel electrophoresis or high-performance liquid chromatography. A protein which is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local alignment algorithm of Smith & Waterman, Adv. Appl. Math. 2:482 (1981), by the global alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). The Smith & Waterman alignment with the default parameters are often used when comparing sequences as described herein.

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403410 (1990), respectively. BLAST and BLAST 2.0 are used, typically with the default parameters, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid (protein) sequences, the BLASTP program uses as defaults a wordlength (W) of 3, an expectation (E) of 10, and the BLOSUM62 scoring matrix (see Henikoff& Henikoff (1989) Proc. Natl. Acad. Sci. USA 89:10915)). For the purposes of this invention, the BLAST2.0 algorithm is used with the default parameters.

The antibodies described herein include humanized antibodies, and chimeric antibodies. A “humanized antibody” refers to an antibody that comprises a donor antibody binding specificity, i.e., the CDR regions of a donor antibody, typically a mouse monoclonal antibody, grafted onto human framework sequences. A “humanized antibody” as used herein binds to the same epitope as the donor antibody and typically has at least 25% of the binding affinity. An exemplary assay for binding affinity is described in Example 5. Methods to determine whether the antibody binds to the same epitope are well known in the art, see, e.g., Harlow & Lane, Using Antibodies, A Laboratory Manual, Cold Spring Harbor Laboratory Press, 1999, which discloses techniques to epitope mapping or alternatively, competition experiments, to determine whether an antibody binds to the same epitope as the donor antibody. A humanized antibody that comprises a novel framework region provided in the invention. The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, and the like) with the desired specificity, affinity, and capability while the constant regions are homologous to the sequences in antibodies derived from another (usually human) to avoid eliciting an immune response in that species.

In one aspect, the splicing incorporation of an exon normally repressed by TDP-43 results from a TDP-43 loss of function.

In another aspect, TDP-43 loss of function generates exons fused-in-frame with a translational reading frame to produce neoepitopes.

In one embodiment, the invention provides a method of detecting TDP-43 loss of function in a subject including detecting in a sample from the subject the presence of a cryptic exon-encoded neoepitope, wherein the cryptic exon-encoded neoepitope is an epitope resulting from a splicing incorporation of an exon normally repressed by TDP-43, and wherein the cryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2), thereby detecting TDP-43 loss of function in the subject.

In one aspect, the cryptic exon-encoded neoepitope is within HDGFL2. In another aspect, the sample is a biological fluid. In various aspects, the biological fluid is selected from the group consisting of blood, cerebrospinal fluid (CSF), saliva, sputum, urine or another biofluid. In one aspect, detecting the presence of a cryptic exon-encoded neoepitope is by enzyme-linked immunosorbent assay (ELISA), protein immunoprecipitation, immunoelectrophoresis, western blot, protein immunostaining, high-performance liquid chromatography (HPLC), or liquid chromatography-mass spectrometry (LC/MS).

Presented below are examples discussing antibodies that specifically bind to cryptic neoepitopes resulting from TDP-43 loss of function, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### A Fluid Biomarker Reveals Loss of Tdp-43 Splicing Repression in Pre-Symptomatic Als: Methods

A fluid biomarker for the pre-symptomatic or prodromal phases of amyotrophic lateral sclerosis (ALS) to enable earlier diagnosis and to facilitate patient recruitment and monitor target engagement in clinical trials is a great unmet need. A central pathological hallmark of the amyotrophic lateral sclerosis-frontotemporal dementia (ALS-FTD) disease spectrum is the nuclear mislocalization and cytoplasmic aggregation of an RNA-binding protein named TAR DNA-binding protein 43 kDa (TDP-43). While a gain-of-function mechanism due to TDP-43 cytoplasmic aggregates has been proposed to contribute to neurodegeneration, emerging evidence supports the idea that loss of TDP-43 splicing repression resulting from depletion of nuclear TDP-43 drives neuron loss in ALS-FTD. TDP-43 pathology can currently only be revealed postmortem, so while such TDP-43 functional deficits are well-documented in end-stage tissues, the extent to which loss of TDP-43 splicing repression occurs during the early stages of disease is unclear. Clarifying this question would provide critical insight into disease mechanisms and inform therapeutic strategies designed to attenuate neuron loss in ALS-FTD.

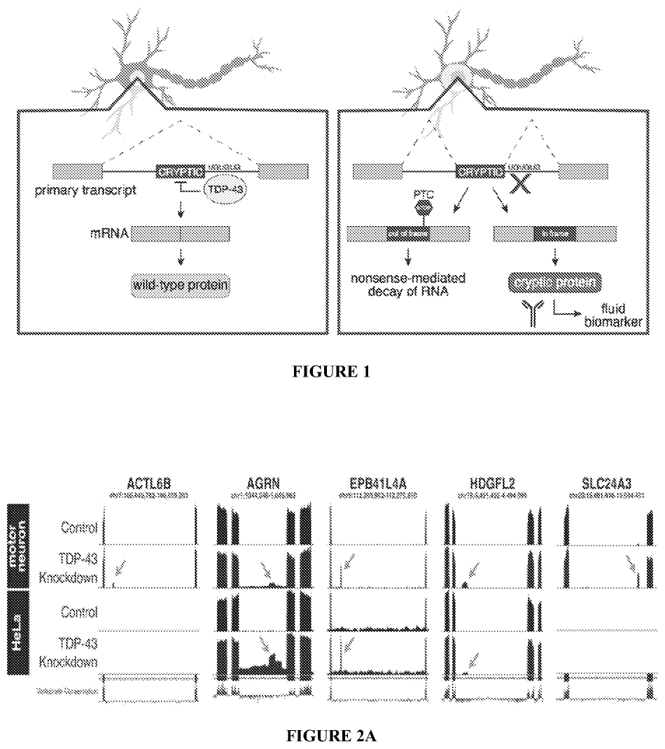

Loss of TDP-43 splicing repression leads to the inclusion of numerous nonconserved cryptic exons, of which ˜3% produce in-frame neoepitopes. It was hypothesized that detecting peptides encoded by cryptic exons in biofluids could reveal how early TDP-43 splicing repression is dysregulated in patients with ALS or FTD and could establish fluid biomarkers that reflect TDP-43 dysfunction (FIG. 1). To test this idea, certain cryptic neoepitopes were selected for antibody generation based on RNA expression data and protein structure modeling. These novel monoclonal antibodies were then validated in HeLa cells depleted of TDP-43 by siRNA. The study then focused on one antibody that was able to reliably detect a cryptic exon-encoded neoepitope in Hepatoma-Derived Growth Factor-Like protein 2 (HDGFL2). Using this novel antibody, a highly specific and sensitive sandwich enzyme-linked immunosorbent assay (ELISA) was developed to determine the dynamic nature of this cryptic peptide target in CSF from pre-symptomatic and symptomatic individuals with familial C9ORF72-linked ALS-FTD.

### Methods:

### Analysis of RNA-Seq Data

Fastq files were downloaded from the NCBI's Sequence Read Archive and aligned to the GRCh38 human genome assembly using STAR (version 2.7.10a) with default parameters. Megadepth was used to convert the output BAM files to BigWig files, and the data were then displayed on the UCSC Genome Browser (http://genome.ucsc.edu/) to visualize the cryptic exons. The data table containing NAUC information was downloaded from ASCOT. Data was subset to the genes of interest, and a heatmap was generated using ggplot2 package in R.

### Protein Structure Generation and Comparison

The cryptic peptide sequences were identified by translating the cryptic exon sequences. The predicted wild-type protein structures were generated using the AlphaFold Monomer v2.0 pipeline and downloaded from the AlphaFold Protein Structure Database. The predicted cryptic protein structures were generated by entering the amino acid sequences of interest into the AlphaFold Monomer v2.0 pipeline (version 2.2.0).

### Generation of Monoclonal Antibodies

Monoclonal antibodies were generated by CDI Laboratories, Inc. Mice were immunized with the cryptic peptide of interest, and hybridomas were produced from these mice. IgG-positive hybridomas were identified by ELISA. Hybridoma lines that produced antibodies recognizing their cognate antigen as the top target on HuProt human protein microarray, which contains >19,500 affinity-purified recombinant human proteins, were identified. Promising hybridoma lines were further screened by protein blot.

### Generation of Wild-Type and Cryptic HDGFL2 Expression Vectors

The wild-type HDGFL2 mRNA sequence (ENST00000616600.5) was identified using UCSC Genome Browser. RNA-sequencing visualization of TDP-43 knockdown motor neurons on UCSC Genome Browser was used to extract the cryptic exon sequence. Codons corresponding to a glycine serine linker and 6-HisTag (GGGSHHHHHH, SEQ ID NO:17) were added to the 3′ end of the sequence directly before the stop codon. A Kozak sequence was added to the 5′ end of the sequence. Codons were optimized by using IDT Codon Optimizer and modifying codons that led to nucleic acid repeats of four or more while retaining the amino acid translation. Restriction sites corresponding to Nsil, XmnI, BclI, BstXI, Nhel, BspEI, XhoI, Xbal, PspOMI, BgIII, NotI, BamHI were removed from the sequence. The resulting sequence was synthesized into the pTwist CMV Puro expression vector by Twist Bioscience.

### RT-PCR Analysis

RNA was extracted from HeLa samples using TRIzol (Life Tech., 15596-026) and RNeasy Mini Kits (Qiagen, 74104). cDNA was derived from total RNA using ProtoScript II First Strand cDNA Synthesis Kit (NEB, E6560S). Numerous primers were designed against cryptic exon targets and screened to identify primer pairs that minimized background bands.

### Protein Blot and Immunoprecipitation-Protein Blot Analysis

Immunoprecipitation was performed by overnight incubation of HeLa lysates with the novel #1-69 antibody against cryptic HDGFL2 at 4° C. with rotation. Then a 50% slurry of Protein G agarose beads (Cell Signaling, #37478) in RIPA lysis buffer was added, and this mixture was incubated with rotation at 4° C. for 1-3 hours. Bead complexes were denatured in NuPAGE LDS Sample Buffer (4×) at 70° C. for 10 minutes and microcentrifuged at 14,000 g for 1 minute. Samples were then analyzed by protein blot.

Protein blot analysis was following electrophoresis using NuPAGE 4-12% Bis-Tris polyacrylamide gels. Proteins were transferred onto PVDF membranes using the iBlot™ Dry Blotting System from Invitrogen. After blocking of the membrane, primary antibodies were incubated overnight at 4° C. with rocking.

### Generation of Hybridomas

Human TDP-43-associated cryptic exons in HeLa cells treated with TDP-43 small interfering RNA (siRNA) were previously identified (Ling et al., 2015). Some of these cryptic exons were selected as targets for development of novel monoclonal antisera. Antibody-secreting hybridoma cells were generated for each target by CDI Laboratories, Inc. following immunization of mice with cryptic exon-encoded peptides.

### Screening of Monoclonal Antisera

A three-part screening approach was used to evaluate the sensitivity and specificity of the novel monoclonal antisera. First, to validate the specificity of the antisera against their respective cryptic exons, constructs encoding myc-tagged GFP-cryptic exon fusions were generated. Lysates from HEK293 cells transfected with either the GFP-myc-cryptic exon fusion or GFP alone were subjected to protein blot analysis with the monoclonal antisera.

Second, antisera were screened using HeLa cells deficient in TDP-43 due to knockdown by TDP-43 siRNA (siTDP) or control (untransfected) HeLa cells. A reverse transcription polymerase chain reaction (RT-PCR) assay was used to confirm cryptic exon expression in the siTDP HeLa cells compared to control HeLa. Following RT-PCR analysis, lysates from control and siTDP HeLa cells were subjected to protein blot analysis by the monoclonal antisera in order to determine specificity of the antibodies.

Third, antisera were screened using a sandwich enzyme-linked immunosorbent assay (ELISA) that was developed to evaluate detection of the cryptic exon-encoded peptide in siTDP and control HeLa cell lysates and then in human biofluids.

### Meso Scale Discovery ELISA Assay

For the sandwich ELISA using the Meso Scale Discovery (MSD) platform, the antibody (#1-69) against the cryptic exon-encoded peptide target in HDGFL2 served as the capture antibody, and a commercial antibody against the wild-type protein was used as the primary detection antibody (Anti-CTB-50L17.10 antibody produced in rabbit; Prestige Antibodies® Powered by Atlas Antibodies). A species-specific sulfo-tagged antibody (Anti Rabbit Antibody Goat SULFO-TAG Labeled; Meso Scale Discovery) was used as a secondary detection reagent to generate electrochemiluminescence. Assays were conducted on MSD MULTI-ARRAY 96-well SECTOR plates and measured with the MESO QuickPlex SQ 120 MM instrument.

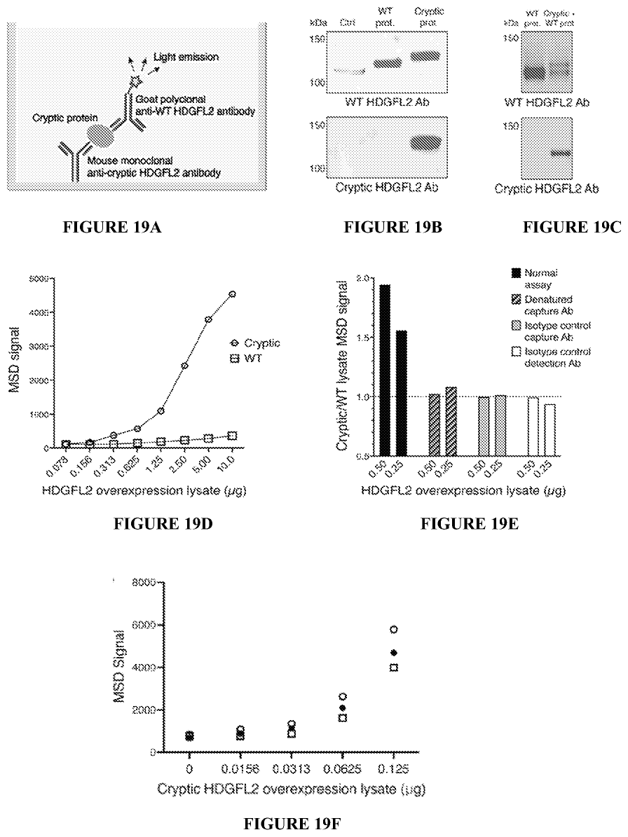

An original pilot study of five C9ORF72-associated ALS and five control subjects utilized duplicates of 120 microliters (μL) of CSF for each individual. In the following studies, CSF samples were assayed with 25 μL of CSF diluted in 75 μL of MSD diluent 35, for a total of 100 μL added per well. A standard curve was constructed using a concentration series of lysate from HeLa cells transfected with the cryptic HDGFL2 expression vector (FIG. 19F). This curve was employed on each plate to normalize signals based on a four-parameter logistic (4PL) fit. In longitudinal assays, all time points of CSF collection for one individual were assayed on the same plate. Immediately before use, CSF was thawed, vortexed, and briefly spun down. Unless otherwise stated, C9ORF72 CSF used was freshly thawed without any previous freeze-thaw cycles. Upon normalization, one C9ORF72 CSF sample was below the lower limit of detection and thus was excluded from further analyses. Any samples previously thawed one time were normalized based on the cryptic HDGFL2 signal differences between a previously thawed aliquot and a previously unthawed aliquot of a set of C9ORF72 CSF samples (FIG. 1).

### Cerebrospinal Fluid Samples

CSF samples from C9ORF72 mutation carriers were provided by the Natural History and Biomarker Study of C9ORF72 ALS (protocol 13-N-0188) with enrollment/study period: 2013-2020 (Offit et al., 2020; PMID 32312103). CSF samples from sporadic ALS subjects and from healthy and non-ALS neurological disease control subjects were provided by the Northeast Amyotrophic Lateral Sclerosis (NEALS) Biorepository. Additional control CSF samples were obtained from individuals with hydrocephalus at Johns Hopkins Bayview Medical Center. Phosphorylated neurofilament heavy chain (pNfH) levels in CSF were measured by Biogen using the ProteinSimple Ella microfluidic immunoassay according to the manufacturer's instructions. Neurofilament light chain (NfL) levels in CSF were measured by Biogen using the Quanterix Simoa assay.

In longitudinal assays, cryptic HDGFL2 was measured in CSF from lumbar punctures at multiple time points in individuals' disease progression and phosphorylated neurofilament heavy chain (pNFH) measurements were evaluated from the same samples performed by Biogen, Inc. Two time points of CSF from 14 pre-symptomatic individuals were analyzed. One pre-symptomatic individual only had pNFH measured at one time point, and another did not have pNFH measurements for either time point, but the other 12 cases had pNFH measurements corresponding to each of the two lumbar punctures. Of the 17 symptomatic individuals from whom multiple time points of CSF were received, 16 had pNFH measurements at both CSF time points, and one had pNFH measurements from three different time points. One individual was diagnosed with mild cognitive impairment (MCI) at their earlier lumbar puncture, so this time point was not included in longitudinal analysis of measurements corresponding to symptomatic ALS, ALS-FTD, or FTD.

### Analysis

Statistics were performed in Stata 17 and GraphPad Prism.

### Example 2

### Development of Monoclonal Antibody Specific to Human TDP-43-Associated Cryptic Exon-Encoded Neo-Epitope

To address whether loss of TDP-43 function occurs during early-stage ALS, a highly sensitive and specific ELISA assay for detection of cryptic exon-encoded peptides in biofluids of patients was developed. A set of human cryptic exons resulting from loss of TDP-43 splicing repression that are fused in-frame to their respective transcripts were identified (Table 1). In addition to other cryptic exon targets within this set (Table 2), several lines of monoclonal antisera directed against a cryptic exon-encoded epitope within hepatoma-derived growth factor-like protein 2 (HDGFL2), a histone-binding protein that is ubiquitously expressed, including in the brain and spinal motor neurons were developed.

TABLE 1
Amino acid sequence of TDP-43 cryptic
exon-encoded peptide. The AGRN cryptic target
was split into three peptides.

| Cryptic | | |
|---|---|---|
| target | Amino acid sequence | SEQ ID NO: |
| HDGFL2 | EPTIWFGKGHSGMLASEGR | SEQ ID NO: 1 |
| | EAVLTRLHESERVRKQERE | |
| | RDTEERRE | |
| ACTL6B | ASFELLGSSNPPASASQSA | SEQ ID NO: 2 |
| | GIIG | |
| ARHGAP32 | GEHLLNSGFEAWKPSP | SEQ ID NO: 3 |
| EBP41L4A | DPCKLKEEITSDIESPYKT | SEQ ID NO: 4 |
| | EVTKGQAEVCESVCAYVYQ | |
| | FFLQVKQD | |
| SLC24A3 | DATVVLLKKGRKEGIRKEE | SEQ ID NO: 5 |
| | WMDGWIANFHRKASVIM | |
| CDO1 | WAMYAKFDQYSRGRGLQFV | SEQ ID NO: 6 |
| | VGGGSGGGWLWYTRNLVDQ | |
| | GN | |
| AGRN (1) | CTELRVRDLWMLCV | SEQ ID NO: 7 |
| AGRN (2) | CVPGSAFTDMFL | SEQ ID NO: 8 |
| AGRN (3) | PGLLWELGSGRLSGD | SEQ ID NO: 9 |
| IGLON5 | QPRAGPSHSRSPRSSSRPS | SEQ ID NO: 10 |
| | SLSAWCQLHRLRMPSPHSL | |
| | ALVQPAGHGLQRDDCVGVT | |
| | EPACCAEVCEWAWAHTCKH | |
| | ALANLSTQCACVDVCVCKG | |
| DNM1 | RPAPPVRVNCHVDFVLSF | SEQ ID NO: 11 |
| AARS1 | ECSGMITAHCSLNFLGSSD | SEQ ID NO: 12 |
| | PLPSSWDYRL | |
| PXDN | VHSDTCDLDKQKWAGETLE | SEQ ID NO: 13 |
| | KRSSDGLELC | |
| NECAB2 | SFSASFCMGGFGHSLSIFE | SEQ ID NO: 14 |
| | SLLCAGHSAR | |

TABLE 2
Generation and purification of monoclonal antibodies against
TDP-43 cryptic exon-encoded peptides indicated in Table 1

| | # of | # of | # of |
|---|---|---|---|
| Cryptic | lines | positive | purified |
| target | screened | lines | antisera |
| HDGFL2 | 24 | 9 | 4 |
| ACTL6B | 56 | 7 | 3 |
| ARHGAP32 | 34 | 5 | 1 |
| EBP41L4A | 50 | 21 | 3 |
| SLC24A3 | 36 | 7 | 4 |
| CDO1 | 14 | 4 | 0 |
| AGRN (1) | 13 | 4 | 2 |
| AGRN (2) | 16 | 5 | 4 |
| AGRN (3) | 9 | 2 | 1 |

### Selection of TDP-43-Dependent Cryptic Exon Targets

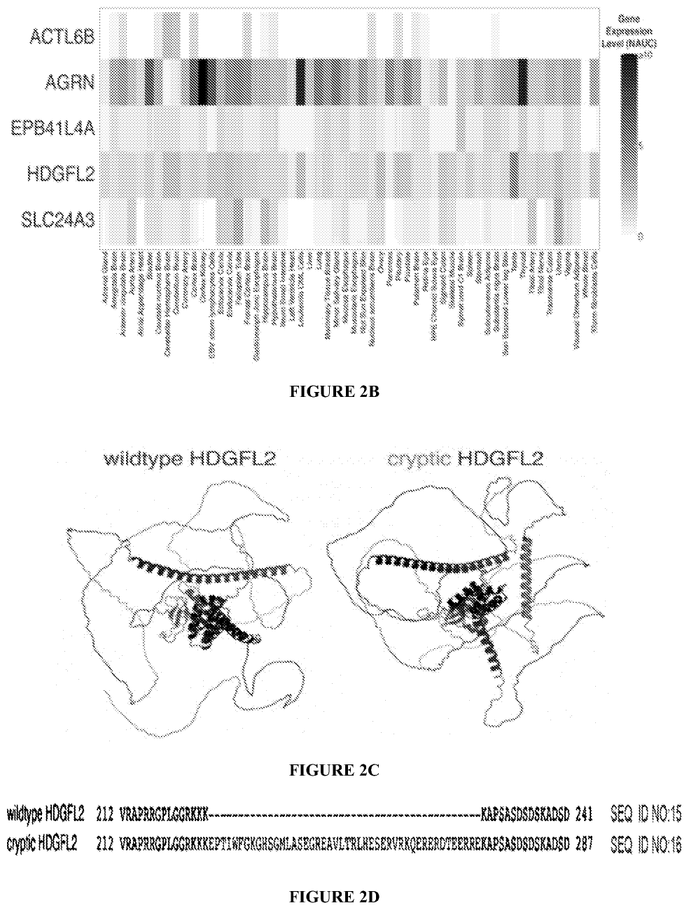

A series of human TDP-43-associated cryptic exons were identified from RNA sequencing of HeLa cells and induced pluripotent stem cell (iPSC)-derived motor depleted of TDP-43 using siRNA. Some of these cryptic exons were selected as targets (FIG. 2A) for development of monoclonal antibodies based on the following criteria: 1) the cryptic exon is spliced in-frame without premature termination codons; 2) the cryptic exon-containing gene is ubiquitously expressed or expressed abundantly in the central nervous system (CNS); and 3) the cryptic exon-encoded peptide contains immunogenic epitopes. While AGRN and HDGFL2 RNA transcripts are ubiquitously expressed, ACTL6B, EPB41L4A, and SLC24A3 are expressed in a more tissue-specific manner. Normalized area under the curve (NAUC) values from ASCOT were used to approximate gene expression levels in different human tissue types.

Genes harboring TDP-43-related cryptic exons were analyzed using alternative splicing catalog of the transcriptome (ASCOT). In-frame cryptic exons in genes with high expression across different human tissues were selected for broad relevance to TDP-43-related diseases. In-frame cryptic exons in genes with high expression in the CNS were also selected due to expected involvement in ALS-FTD (FIG. 2B). Following selection of cryptic exon targets based on the initial two criteria, AlphaFold protein structure prediction software was used to model the cryptic peptide-containing proteins of interest to visualize the accessibility of the cryptic epitopes and predict the preservation of the proteins' native conformations (FIG. 2C). Cryptic exon-encoded epitopes with immunogenic potential that did not significantly disrupt protein conformation or interfere with binding of antibodies targeting wild-type sequences were selected as prospective targets. Inclusion of the cryptic exon in mRNA leads to the addition of 46 amino acids predicted to form an alpha helix structure between flanking unstructured regions. Both structures are generated using AlphaFold predictions derived from amino acid sequences. Wild-type HDGFL2 protein structure can be found on the AlphaFold protein structure database (UniProt: Q7Z4V5).

Of the cryptic exons meeting these criteria, one promising target selected for development of novel monoclonal antibodies was the cryptic exon-encoded epitope within Hepatoma-Derived Growth Factor-Like protein 2 (HDGFL2), a histone-binding protein that is nearly ubiquitously expressed and is detected in brain and spinal motor neurons (FIG. 2B). Mice were immunized with the HDGFL2 cryptic exon-encoded peptide (FIG. 2D), antibody-secreting hybridoma cells were isolated for this target, and antibodies were purified from hybridoma cell line. Cryptic inclusion is 46 amino acids long and did not impact flanking amino acids.

### Monoclonal Antibody Specific to Human TDP-43-Dependent Cryptic Exon-Encoded Neo-Epitope

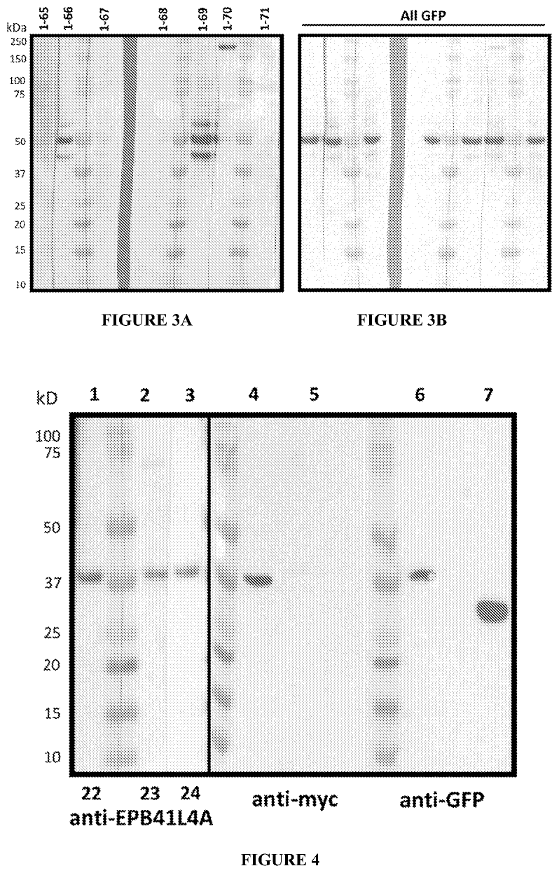

A three-part screening approach was used to evaluate the sensitivity and specificity of the novel monoclonal antibodies. First, monoclonal lines that would recognize the cryptic exon-encoded peptide in HDGFL2 (termed cryptic HDGFL2) when expressed as a myc-tagged green fluorescent protein (GFP)-cryptic exon fusion protein were searched for. Lysates from HEK293 cells transfected with either the GFP-myc-cryptic HDGFL2 fusion or GFP alone were subjected to protein blot analysis with monoclonal antibodies. Of monoclonal lines #1-65 through 1-71 against the cryptic HDGFL2 epitope, lines #1-66 and 1-69 detected with specificity the fusion protein containing the cryptic HDGFL2 peptide (FIGS. 3A-3B and 4). Line #1-69 was used moving forward.

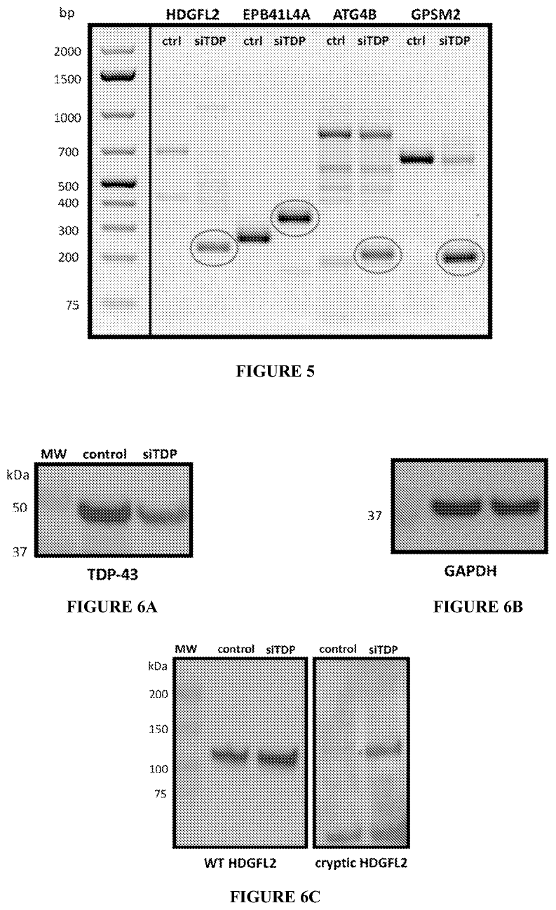

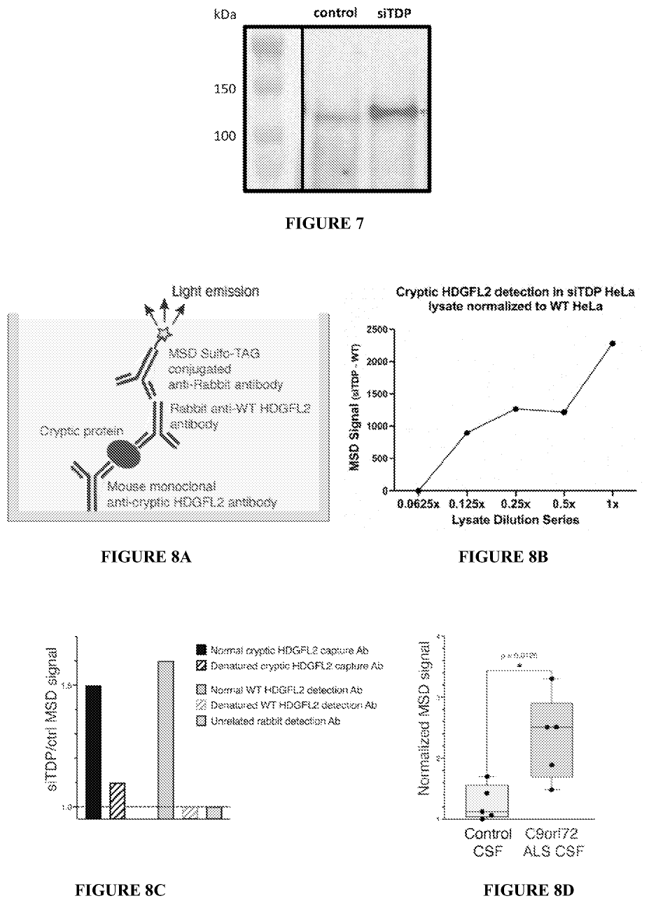

Second, an siRNA knockdown strategy was used in Hela cells to deplete TDP-43 (siTDP HeLa) and screened antibodies in siTDP vs. control HeLa lysates (FIGS. 6A and 6B). It was confirmed that, as expected, the appearance of HDGFL2 containing cryptic exons in siTDP compared to control cells (FIG. 5). Total cell extracts from control and siTDP HeLa cells were then subjected to protein blot analysis with the monoclonal antibodies to determine specificity of the antibodies. As expected, a band corresponding to the normal HDGFL2 protein was observed in both control and siTDP HeLa cells using an antibody recognizing the native HDGFL2 (FIG. 6C). Importantly, the monoclonal #1-69 cryptic antibody recognized a novel band of the expected size, presumably corresponding to the cryptic peptide-containing protein, in extract of HeLa cells depleted of TDP-43 but not control cells (FIG. 6C). To further demonstrate the specificity of the monoclonal antibody, immunoprecipitation (IP) was performed using this antibody followed by protein blot analysis with the antibody recognizing the native HDGFL2. While no positive band of correct molecular weight was observed in control extract, a robust band of expected size for the cryptic exon-encoded peptide within HDGFL2 was evident in siTDP HeLa cells (FIG. 7). Thus, these data demonstrate that this monoclonal antibody specifically recognizes the cryptic exon-encoded peptide within HDGFL2.

Monoclonal lines that would recognize the cryptic exon of interest when expressed as a myc-tagged GFP fusion protein were first screened for. Lysates of HEK293 cells transfected with a construct expressing either GFP-myc-HDGFL2 or GFP alone were subjected to protein blot analysis by monoclonal lines #1-65 through 1-71 against the cryptic HDGFL2 epitope. Lines (#1-66 and 1-69) detected with specificity the fusion protein containing the cryptic HDGFL2 peptide (FIGS. 3A and 3B). This strategy was used with the other cryptic exon targets as well in order to screen the monoclonal lines (FIG. 4).

To test whether these antisera recognize selectively HDGFL2 containing the “cryptic” peptide, an siRNA knockdown strategy was used in HeLa cells to deplete TDP-43. As expected, the appearance of a series of previously identified RNAs containing cryptic exons, was confirmed including GPSM2, ATG4B and EBP41L4A, in siTDP compared to control cells (FIG. 5). To validate that these antisera specifically recognize the cryptic exon-encoded peptide within HDGFL2, total cell extracts from control HeLa and HeLa cells depleted of TDP-43 were subjected to protein blot analysis. As expected, a band corresponding to the normal protein was observed using a commercial antibody recognizing this native HDGFL2 in control and HeLa cells lacking TDP-43. Importantly, these monoclonal antisera recognize a novel band of the expected size (presumably corresponding to the cryptic peptide containing protein) in extract of HeLa cells depleted of TDP-43, but not control HeLa cells (FIGS. 6A-6C). Thus, these data indicate that these monoclonal antisera selectively recognize the cryptic exon-encoded peptide within HDGFL2. To further demonstrate the specificity, immunoprecipitation (IP) was performed using these antibodies followed by protein blot analysis using an antibody recognizing the native HDGFL2. While no positive band of correct molecular weight was observed in control extract, a robust band of expected size for the cryptic exon-encoded peptide within HDGFL2 was evident in HeLa cell extract depleted of TDP-43 (FIG. 7). Thus, these data demonstrate that these monoclonal antibodies specifically recognize the cryptic exon-encoded peptide within HDGFL2.

A highly sensitive MSD sandwich ELISA for detection of TDP-43-dependent cryptic peptide

To employ the novel monoclonal antibodies to detect cryptic exon-encoded peptides in human CSF, a highly sensitive sandwich ELISA was developed using the Meso Scale Discovery (MSD) platform and validated this assay for the cryptic exon target in HDGFL2. In the same manner as IP-protein blot analysis was performed, the #1-69 cryptic monoclonal antibody was used as the capture (coating) antibody to pull down the cryptic exon-encoded peptide within HDGFL2 and the rabbit antibody recognizing the wild-type (WT) HDGFL2 as the primary detection antibody. The amount of cryptic HDGFL2 can be quantified indirectly by using a sulfo-tagged anti-rabbit antibody for secondary detection (FIG. 8A). This MSD ELISA protocol was tested using the control and siTDP HeLa lysates. Compared to control lysate, a concentration-dependent increase in MSD signal was observed in siTDP HeLa lysate (FIG. 8B). A dose-dependent increase of MSD signal in siTDP HeLa lysate compared to control HeLa lysate. MSD signal of control HeLa was subtracted from that of siTDP. When the primary detection antibody against WT HDGFL2 was replaced with an unrelated rabbit antibody, no difference between MSD signal of siTDP and control HeLa lysates was observed (FIG. 8C), indicating that the siTDP signal is specific. When the primary detection antibody against WT HDGFL2 was denatured by heating at 95° C. for 30 minutes prior to addition to the plate, there was again no difference between MSD signal of siTDP and control HeLa lysates (FIG. 8C). These data demonstrate the specificity of the assay for HDGFL2 binding by the detection antibody. When the cryptic HDGFL2 capture antibody was denatured by heating at 95° C. for 30 minutes prior to addition to the plate, the ratio of siTDP/control HeLa MSD signal decreased to control level (FIG. 8C). These data support the specificity of the assay for cryptic HDGFL2 binding by the capture antibody. Taken together, these data establish a highly sensitive and specific sandwich ELISA for the detection of cryptic peptide-containing protein, a valuable method to monitor TDP-43 loss of function in biofluids of ALS patients.

To develop a highly sensitive ELISA assay in effort to detect cryptic peptide containing proteins in biofluids of ALS, including C9orf72 patients, the Meso Scale Discovery (MSD) platform was employed. In the same manner as IP-protein blot analysis was performed, the “cryptic” monoclonal antibody as the capture (coating) antibody was used to pull down the cryptic exon-encoded peptide within HDGFL2 (termed cryptic HDGFL2) from HeLa extract depleted of TDP-43 and the rabbit antisera recognizing the native HDGFL2 as the secondary (detection) antibody. The amount of cryptic HDGFL2 can be quantified indirectly by using the Sulfo-tagged anti-rabbit antisera. Using the 1-69 cryptic antibody, this MSD ELISA protocol was then tested using the control and siTDP HeLa lysates. Compared to control lysate, a significant increase in MSD signal was observed in HeLa lysate depleted of TDP-43 (FIGS. 8A-8C). These data establish a highly sensitive and specific ELISA assay for the detection of cryptic peptide-containing protein, a valuable method to monitor level of TDP-43 loss in biofluids of ALS patients.

### Example 3

### Detection of Cryptic Hdgfl2 in Pre-Symptomatic Csf of C90Rf72 Mutation Carriers

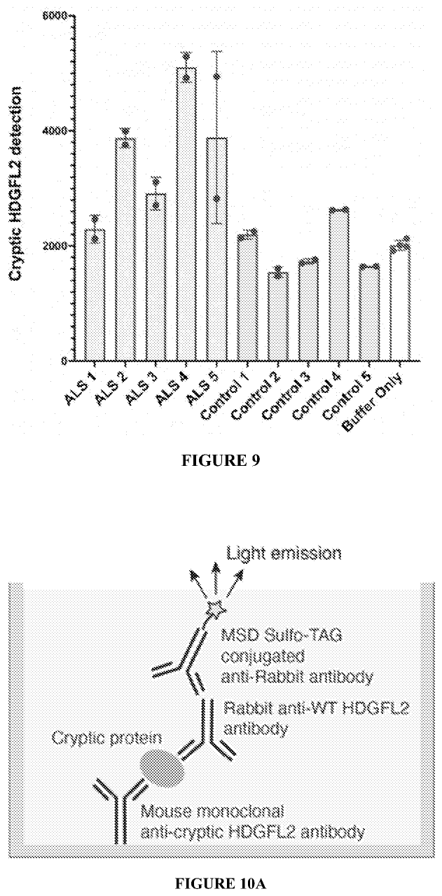

In a pilot study using CSF of C9orf72 mutation carriers with ALS and control individuals, the MSD signal for cryptic HDGFL2 was significantly elevated in ALS samples (mean=1601) as compared to those of controls (mean=−64.10; p<0.013; FIGS. 8D and 9A).

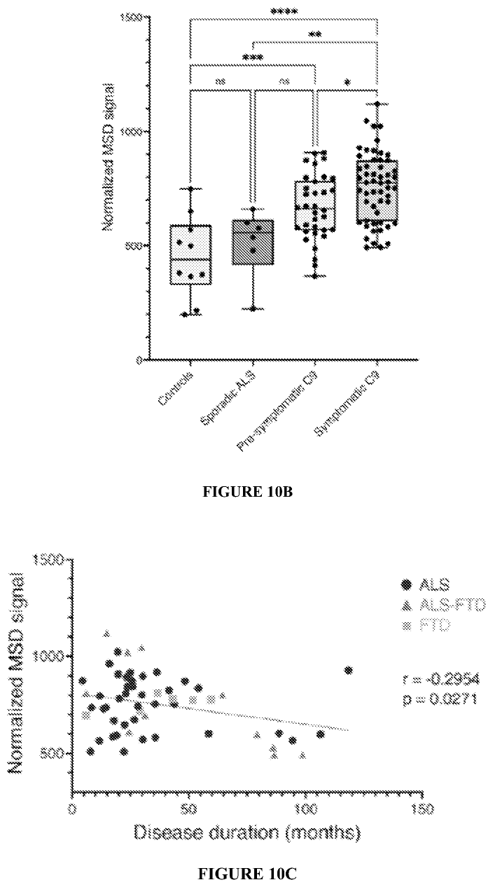

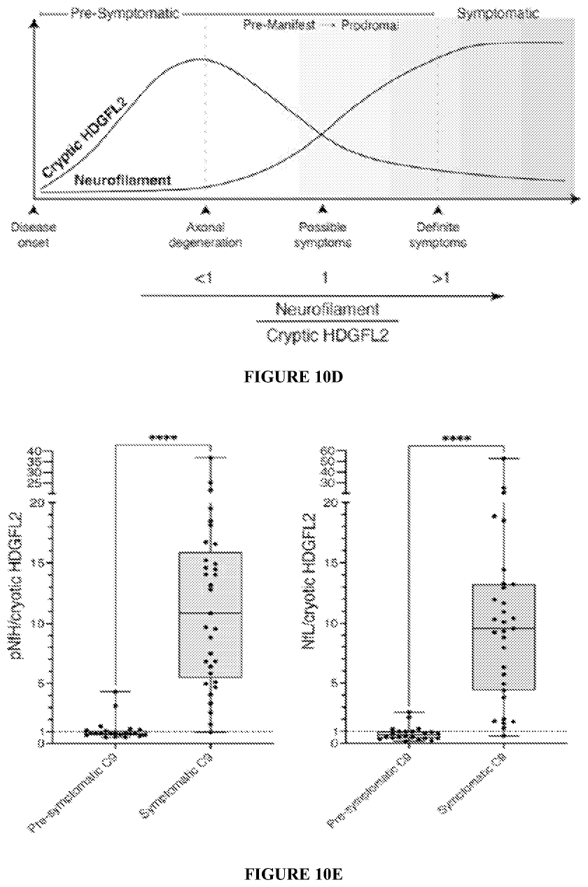

The study was then expanded into a larger cohort of 43 C9orf72 mutation carriers. This cohort included 27 individuals with symptomatic ALS, ALS-FTD, or FTD; one individual with MCI; and 15 pre-symptomatic individuals. The average age of this cohort was 52.57 years old (range=30.16-75.52, standard deviation=10.97), and the cohort was 48.84% (21/43) female. There was no correlation between age and CSF levels of cryptic HDGFL2 detected by the MSD assay (Pearson correlation, r=−0.0452, p=0.7737), and there was no significant difference in cryptic HDGFL2 detection between male and female individuals (two-sample t-test, p=0.3464). Individuals' levels of cryptic HDGFL2 did not correlate with their scores on the Revised Amyotrophic Lateral Sclerosis Functional Rating Scale (ALSFRS-R) (Pearson correlation, r=0.1246, p=0.4260). However, among the 27 individuals who had phenoconverted to symptomatic ALS (n=20), ALS-FTD (n=5), or FTD (n=2), levels of cryptic HDGFL2 showed a correlation nearing significance with symptom duration (Pearson correlation, r=−0.3679, p=0.0590; FIG. 10C), suggesting that levels of cryptic HDGFL2 tend to be higher during the earlier stage of symptomatic disease.

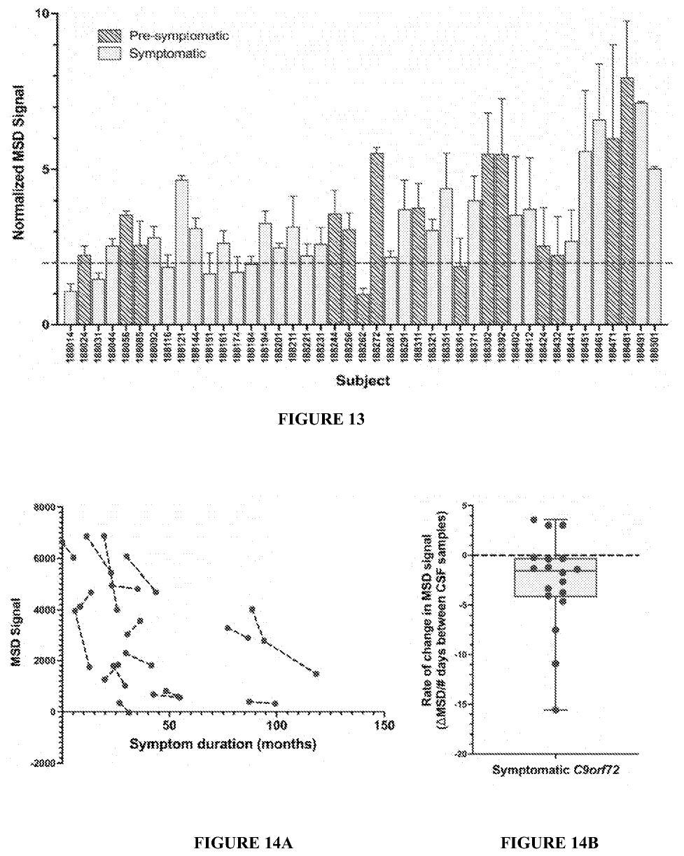

This cohort of C9orf72 mutation carriers offered a unique opportunity to assay pre-symptomatic individuals, as subjects were recruited due to their mutation status, but not all had phenoconverted to symptomatic disease yet. CSF of 15 pre-symptomatic individuals were assayed, along with the 27 individuals with symptomatic ALS, ALS-FTD, or FTD and one individual with MCI, and it was found that elevated levels of cryptic HDGFL2 were detectable in CSF of several pre-symptomatic individuals, with some of the highest signals detected occurring in pre-symptomatic cases (FIG. 13). The mean MSD signal was higher in the pre-symptomatic group (1877, standard deviation=941) compared to the symptomatic group (1609, standard deviation=753), but this difference was not statistically significant (two-tailed t-test, p=0.3179).

### Presence of Cryptic HDGFL2 in CSF of Pre-Symptomatic C9ORF72 Mutation Carriers

In a pilot study using CSF of C9ORF72 mutation carriers with ALS and control individuals, the MSD signal for cryptic HDGFL2 was significantly elevated in ALS samples (mean=1601) as compared to those of controls (mean=−64.10; p<0.013; FIG. 8D). The study was then expanded into a larger, longitudinal cohort of 46 C9ORF72 mutation carriers with a total of 88 CSF samples, with each individual contributing one, two, or three time points of CSF. This cohort of C9ORF72 mutation carriers offered a unique opportunity to assay pre-symptomatic individuals, as subjects were recruited due to their mutation status, but not all had pheno-converted to symptomatic disease yet. The study cohort included 31 individuals with symptomatic ALS (n=20), ALS-FTD (n=8), or FTD (n=3) and 15 pre-symptomatic individuals. The mean age was 52.0 years old (range=28.6-72.6, standard deviation [SD]=11.4), and 47.8% (22/46) were female.

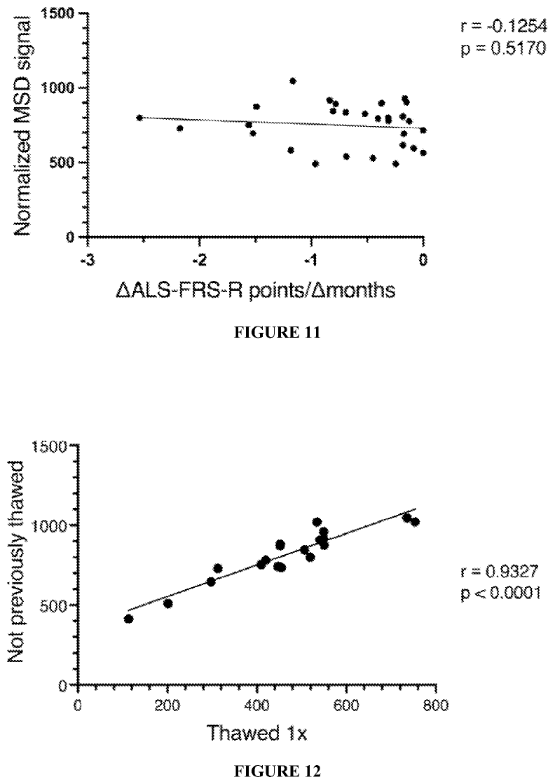

Normalized cryptic HDGFL2 MSD signals in males (mean=759.1) were significantly higher compared to females (mean=689.0, two sample t-test, p=0.03). However, there was no difference between cryptic HDGFL2 MSD signals in symptomatic males (mean=769.9) and symptomatic females (mean=741.4, two sample t-test, p=0.52). Normalized cryptic HDGFL2 signals in both pre-symptomatic (mean=665.4) and symptomatic (mean=758.4) C9ORF72 mutation carriers were significantly higher compared to controls (mean=451.4) (one-way ANOVA with Tukey's multiple comparisons test, FIG. 10B). CSF of 6 sporadic ALS cases were also assayed. While cryptic HDGFL2 signals (mean=513.0) were elevated compared to controls, this difference was not significant. C9ORF72 mutation carriers' levels of cryptic HDGFL2 did not correlate with their scores on the Revised Amyotrophic Lateral Sclerosis Functional Rating Scale (ALSFRS-R) (Pearson correlation, r=−0.11, p=0.31). Additionally, cryptic HDGFL2 signals did not correlate with rate of disease progression as measured by change in ALS-FRS-R score per months elapsed since the prior CSF collection point (FIG. 11; Pearson correlation, r=−0.12, p=0.52). Rate of disease progression was measured as the change in ALS-FRS-R points divided by the number of months that had passed since the prior CSF collection point. However, among the 31 individuals who had pheno-converted to symptomatic ALS, ALS-FTD, or FTD, levels of cryptic HDGFL2 showed a negative correlation with symptom duration (Pearson correlation, r=−0.30, p=0.03; FIG. 10C), suggesting that levels of cryptic HDGFL2 tend to be higher during the earlier stage of symptomatic disease.

### Example 4

### Csf Ratio of Pnfh/Cryptic Hdgfl2 is a Marker for Phenoconversion.

To clarify the dynamics of cryptic HDGFL2 levels during disease progression, longitudinal CSFs from C9ORF72 mutation carriers were assessed, including those who donated at three consecutive time points. In 82.35% (14/17) of symptomatic individuals, levels of cryptic HDGFL2 decreased with disease progression (FIGS. 14A and 14B). The two time points for one individual correspond to a diagnosis of mild cognitive impairment at the first time point and FTD at the second. Another individual converted from ALS to ALS-FTD. All others had diagnoses of ALS (n=10), ALS-FTD (n=4), or FTD (n=1) at each time point.

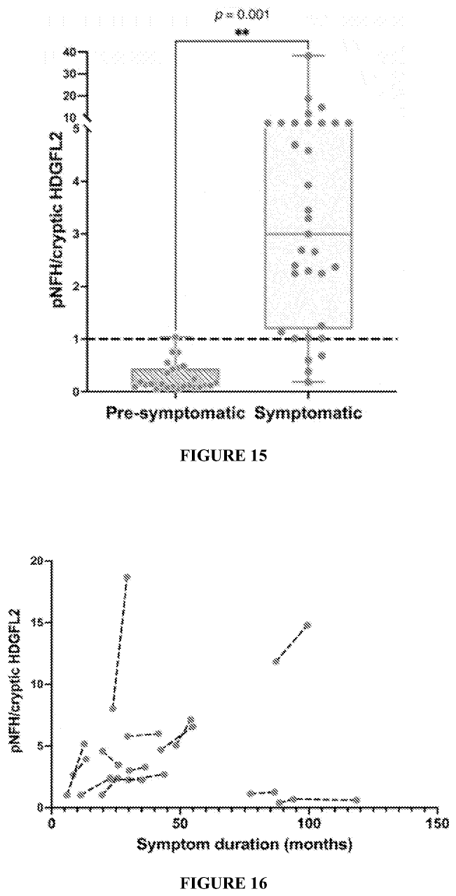

As cryptic HDGFL2 appears to increase during the pre-symptomatic stage of disease and then decrease with symptomatic disease progression, and this trend is in the direction opposite to that of neurofilament, the ratio of phosphorylated neurofilament heavy chain (pNFH) to cryptic HDGFL2 (pNFH/cryptic HDGFL2) was analyzed, anticipating that a ratio less than 1 may indicate a pre-symptomatic stage of disease, while a ratio greater than 1 would indicate a symptomatic stage. For each of the longitudinal samples, the ratio of pNFH to cryptic HDGFL2 was determined. Of the 25 pNFH/cryptic HDGFL2 ratios for the pre-symptomatic group, 96% (24/25) were less than 1. Of the 34 pNFH/cryptic HDGFL2 ratios for this symptomatic group, 88.24% (30/34) were greater than 1 (FIG. 15). As shown in FIG. 15, pNFH values were available for two lumbar punctures of 12 pre-symptomatic individuals and for one lumbar puncture of a 13th pre-symptomatic individuals. pNFH values were available for two lumbar punctures of 16 symptomatic individuals and for three lumbar punctures of a 17th individual. One symptomatic individual had a negative MSD value (−30) after subtracting the background signal. In order to determine a ratio, this value was converted to 1 for mathematical purposes. The point for this ratio (>1) is not shown, as it is an outlier. Another individual was diagnosed with MCI at their earlier lumbar puncture, so this time point was not included with the other measurements corresponding to symptomatic ALS, ALS-FTD, or FTD. Of the 25 pNFH/cryptic HDGFL2 ratios for the pre-symptomatic group, 96% (24/25) were less than 1. Of the 34 pNFH/cryptic HDGFL2 ratios for the symptomatic group, 88.24% (30/34) were greater than 1. These data support the idea that the ratio of pNFH/cryptic HDGFL2 in CSF of ALS patients is a determinant for phenoconversion and indicate that the loss of TDP-43 function occurs during early-stage disease.

Because pNFH levels tend to plateau during symptomatic disease while the data suggest that levels of cryptic HDGFL2 decrease, one would expect the ratio of pNFH/cryptic HDGFL2 to increase throughout symptomatic disease progression. In 87.5% (14/16) of symptomatic individuals who had diagnoses of ALS, ALS-FTD, or FTD at each lumbar puncture, pNFH/cryptic HDGFL2 increased across disease progression (FIG. 16). As shown in FIG. 16, ratios of pNFH/cryptic HDGFL2 in longitudinal samples from 15 C9orf72 mutation carriers who were symptomatic at each of the two or three time points in disease. One individual is not represented in the figure due to a large ratio resulting from cryptic HDGFL2 detection below the background signal, but this ratio also increased between lumbar punctures. The individual who had three lumbar punctures available showed an increased ratio of pNFH/cryptic HDGFL2 between the first two lumbar punctures, but this ratio decreased slightly (−0.08) between the second and third lumbar punctures.

### Example 5

### Discussion

While it has been proposed that loss of TDP-43 splicing repression of cryptic exons underlies disease pathogenesis of ALS-FTD, evidence that such loss of TDP-43 function occurs during early-stage disease, rather than being an end-stage phenomenon, remains elusive. The findings demonstrating detection of cryptic HDGFL2 in CSF of ALS patients now provide direct evidence that loss of TDP-43 splicing repression occurs during early-stage disease, including the pre-symptomatic phase (FIG. 9). The analysis of accumulation of cryptic exon-encoded epitope in CSFs for numerous C9ORF72-linked individuals in a cross-sectional study as well as some patients from several points along their disease course in a longitudinal study allows not only to establish loss of TDP-43 splicing repression occurring at a pre-symptomatic stage but also to validate the dynamic nature of TDP-43 cryptic proteins in which the ratio of pNFH to cryptic HDGFL2 can be employed as an ALS staging tool. It is thus proposed a staging scale in which a ratio of pNFH/cryptic HDGFL2 less than 1 indicates a pre-symptomatic stage of disease, while a ratio greater than 1 indicates a symptomatic stage of disease, indicating the ability to predict phenoconversion in ALS. If confirmed with other TDP-43 cryptic targets in the future, these findings will further establish that loss of TDP-43 splicing repression occurs during the pre-symptomatic stage of disease and provide strong rationale to develop therapeutic strategies to complement TDP-43 splicing repression deficits—for example, an AAV9 gene therapeutic strategy that expresses a splicing repressor, termed CTR for ALS. In addition, these findings will also provide a strategy to develop an ALS staging tool for the prodromal phase of ALS.

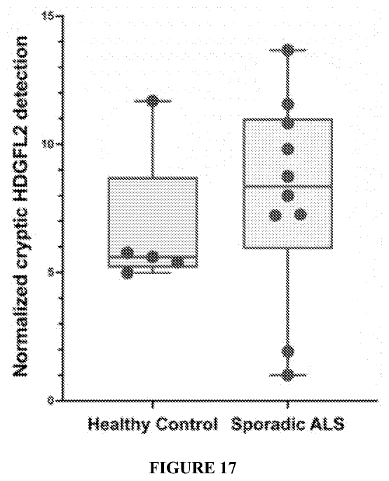

Although the findings reported here utilized CSF of C9ORF72 mutation carriers representing familial ALS, relevance of cryptic peptide biomarkers to sporadic ALS was shown by displaying elevated cryptic HDGFL2 in CSF of individuals with sporadic ALS compared to healthy controls (FIG. 17). Sporadic ALS makes up 90-95% of ALS cases, so extending this study of cryptic peptide biomarkers into sporadic ALS is important.

A highly sensitive multiplexed MSD assay will be developed so that cryptic HDGFL2 and other disease-relevant markers, such as NFs, tau, amyloid-β and α-synuclein can be measured simultaneously, providing additional insight into disease staging for ALS and other human diseases exhibiting TDP-43 pathology. This multiplexed system will also allow to analyze several cryptic peptides at once. It is envisioned that the ability to detect a set of TDP-43 cryptic exon targets which may display different dynamics throughout disease progression. Analyzing the changes of these targets throughout disease could provide detailed information on disease staging, progression, and potentially prognosis that is not currently available for ALS-FTD.

Currently, TDP-43 pathology is revealed only at autopsy. In this study, it was shown that antibodies against cryptic exon-encoded peptides can serve as biomarkers for loss of TDP-43 function, which appears to occur pre-symptomatically in familial ALS. Identifying patients earlier in disease would facilitate prompt recruitment to clinical trials and may provide therapeutics with a greater chance of success. Because detection of cryptic exon-encoded peptides in patient biofluids reflects loss of TDP-43 function, evaluating the dynamics of cryptic peptide biomarkers could provide a way of measuring target engagement for new therapeutics aimed at restoring TDP-43 function. Notably, the impact of this work extends beyond the ALS-FTD spectrum. As many cases of Alzheimer's disease (AD) also possess TDP-43 pathology, cryptic peptide biomarkers could distinguish “pure AD” from mixed etiology dementia, which likely warrant different treatment strategies. Several other conditions involve TDP-43 proteinopathy, including Inclusion Body Myositis, multiple sclerosis and chronic traumatic encephalopathy, so the benefits of these biomarkers could be far-reaching.

### Example 6

### Localization of Tdp-43-Dependent Cryptic Hdgfl2 in Neurons of Als-Ftd Brains

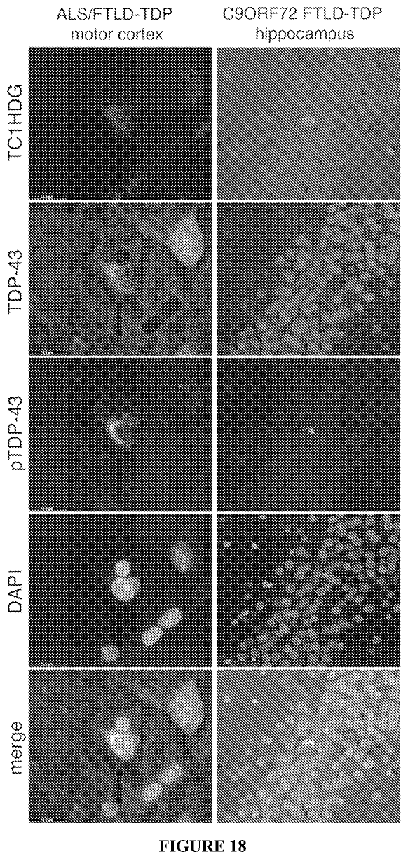

The TC1HDG antibody was used to examine whether cryptic HDGFL2 can be found in neurons of cases of ALS-frontotemporal lobar degeneration (FTLD). In both ALS/FTLD-TDP motor cortex and C9ORF72-linked FTLD-TDP hippocampus, TC1HDG immunoreactivity is seen in nuclei of neurons with reduced nuclear TDP-43 immunoreactivity and with phosphorylated TDP-43-immunoreactive cytoplasmic aggregates (FIG. 18). These results support the prediction that HDGFL2 mRNA containing cryptic exon can be translated in neurons in which TDP-43 has been depleted from the nucleus and that cryptic HDGFL2 is detectable in brains of human disease. Cryptic HDGFL2 was detected by monoclonal antibody (TC1HDG) in neurons that were depleted of nuclear TDP-43 and contained phosphorylated TDP-43 (pTDP-43) cytoplasmic aggregates.

### Example 7

### a Highly Sensitive Msd Sandwich EELISA for Detection of Tdp-43-Dependent Cryptic Peptide

To employ the monoclonal antibodies to detect cryptic exon-encoded peptides in human CSF, a highly sensitive sandwich ELISA was developed using the Meso Scale Discovery (MSD) platform and validated this assay for the cryptic exon target in HDGFL2. In the same manner as IP-protein blot analysis was performed, the TC1HDG cryptic monoclonal antibody was used as the capture (coating) antibody to pull down the cryptic exon-encoded peptide within HDGFL2 and an antibody recognizing the wild-type (WT) HDGFL2 as the detection antibody. large quantities of goat antibody (gTEA1.2) recognizing the same immunogen as the previously used rabbit antibody against wild-type HDGFL2 were generated, and the MSD GOLD™ SULFO-TAG NHS-Ester kit was used to conjugate this goat antibody with a sulfo-tag required for generation of quantifiable electrochemiluminescent signal (FIG. 19A).

This MSD ELISA protocol was tested using lysates of HeLa cells transfected to overexpress either cryptic or WT HDGFL2 (FIG. 19B). A band corresponding to cryptic HDGFL2 wass detectable only in HeLa transfected to overexpress cryptic HDGFL2. Compared to WT HDGFL2 overexpression lysate, a concentration-dependent increase in MSD signal was observed in cryptic HDGFL2 overexpression lysate (FIG. 19C). Probing with the TC1HDG cryptic antibody revealed a single band of the expected size in only the cryptic and WT co-overexpression lysate. When the cryptic HDGFL2 capture antibody was denatured by heating at 95° C. for 30 minutes prior to addition to the plate, the ratio of cryptic/WT HDGFL2 lysate MSD signal decreased to 1 (FIG. 19D). Additionally, when the cryptic HDGFL2 capture antibody was replaced with an isotype control, the ratio of cryptic/WT HDGFL2 decreased to 1. These data demonstrate the specificity of the assay for cryptic HDGFL2 binding by the capture antibody. When the detection antibody was replaced with a sulfo-tagged isotype control antibody, there was again no difference between MSD signals of cryptic and WT HDGFL2 overexpression lysates (FIG. 19E). Denatured capture antibody was heated at 95° C. for 30 minutes. Mouse IgG and sulfo-tagged goat IgG were used as isotype controls for capture and detection antibodies, respectively. Taken together, these data establish a highly sensitive and specific sandwich ELISA for the detection of cryptic peptide-containing protein, a valuable method to monitor TDP-43 loss of function in biofluids of ALS patients.

### Example 8

### Presence of Cryptic Hdgfl2 in Csf of Presymptomatic C9Orf72-Mutation Carriers

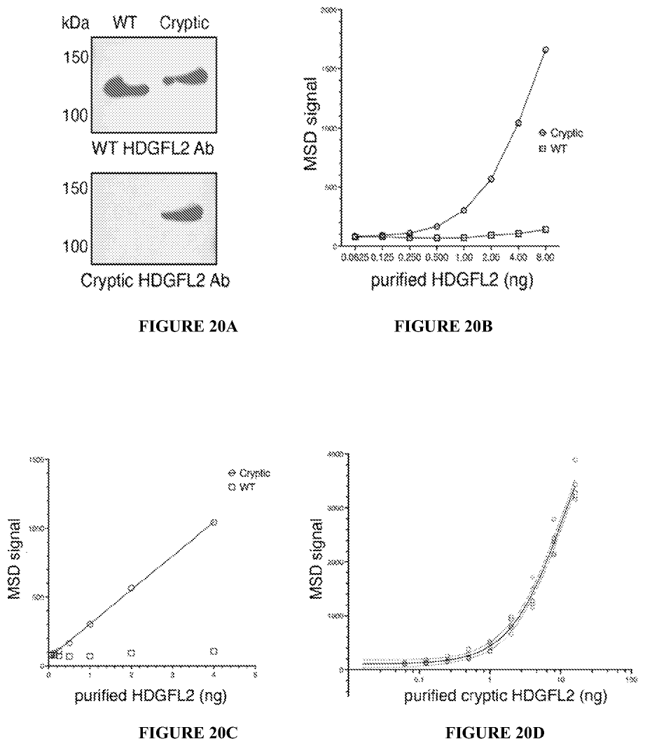

In order to quantify cryptic HDGFL2 in patient biofluids, a standard curve of known concentrations of purified cryptic HDGFL2 was developed, which was included on each MSD ELISA plate (FIGS. 20A-20D). With the MSD ELISA, CSF from a longitudinal cohort of 47 C9ORF72-mutation carriers was analyzed, provided by the National Institute on Aging (NIA; Offit et al., 2020). This cohort included a total of 89 CSF samples, with each individual contributing one, two, or three time points of CSF. This cohort of C9ORF72-mutation carriers offered a unique opportunity to assay both symptomatic and presymptomatic individuals, as subjects were recruited due to their mutation status, but not all had phenoconverted to symptomatic disease yet. Bands of the expected sizes were detectable by a goat antibody against WT HDGFL2 (gTEA1.2) for both proteins. Only cryptic HDGFL2 showed a band of the expected size when probed with the novel TC1HDG cryptic antibody.

The study cohort included 32 individuals with symptomatic ALS (n=21), ALS-FTD (n=8), or FTD (n=3) and 15 presymptomatic individuals. The mean age from all CSF collections was 52.8 years old (range-28.6-72.6, standard deviation [SD]=11.3), and 23/47 (48.9%) of subjects were female. MSD cryptic HDGFL2 levels were not significantly different between males (mean=59.8 ng/mL, SD=348.6) and females (mean=1.9 ng/mL, SD=4.7; two sample t-test, p=0.27), although levels trended higher in males.

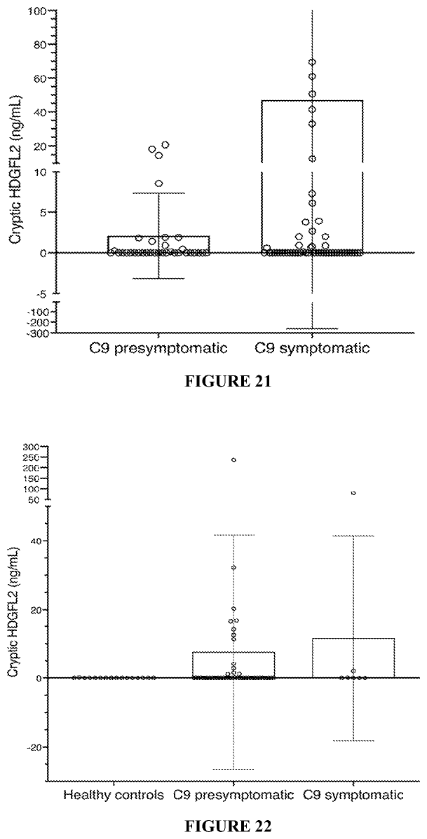

Of the symptomatic group, 20/55 (36.4%) CSF samples had quantifiable levels of cryptic HDGFL2 (range=0.13-2290 ng/ml). Of the presymptomatic group, 12/34 (35.3%) CSF samples had quantifiable levels of cryptic HDGFL2 (range-0.13-21 ng/ml; FIG. 21). One point at 2289.96 ng/mL was not shown for the C9 symptomatic group. Additionally, the upper standard deviation bar is not shown at 355 ng/mL. Of the symptomatic group, 20/55 (36.4%) CSF samples had quantifiable levels of cryptic HDGFL2 (range 0.13-2289.96 ng/ml). Of the presymptomatic group, 12/34 (35.3%) CSF samples had quantifiable levels of cryptic HDGFL2 (range 0.13-21 ng/ml). The rest of the CSF samples had MSD measurements below the lower limit of quantitation. There was no significant difference between mean cryptic HDGFL2 levels of presymptomatic and symptomatic C9ORF72-mutation carriers.

Due to the novel finding that cryptic peptides reflecting TDP-43 loss of function are detectable in presymptomatic disease, an additional cohort of largely presymptomatic C9ORF72-mutation carriers from the Dominant Inherited ALS (DIALS) Network was analyzed in order to further characterize early loss of TDP-43 splicing repression. From this cohort, 16 CSF samples from 12 healthy controls, 49 CSF samples from 42 presymptomatic C9ORF72-mutation carriers, and 7 CSF samples from 6 symptomatic converters were analyzed (FIG. 22). Of the control group, 2/16 CSF samples had quantifiable levels of cryptic HDGFL2 (range=0.015-0.10 ng/ml). Of the symptomatic group, 3/7 CSF samples had quantifiable levels of cryptic HDGFL2 (range=0.094-79.2 ng/ml). Of the presymptomatic group, 17/49 CSF samples had quantifiable levels of cryptic HDGFL2 (range=0.10-236.7 ng/ml). The symptomatic group had the highest levels of cryptic HDGFL2 (mean=11.6, SD=29.8), the presymptomatic group had the second highest levels (mean=7.6, SD=34.1), and the control group had the least (mean=0.007, SD=0.03). Of the control group, 2/16 CSF samples had quantifiable levels of cryptic HDGFL2 (range-0.015-0.10 ng/ml). Of the symptomatic group, 3/7 CSF samples had quantifiable levels of cryptic HDGFL2 (range=0.094-79.2 ng/mL). Of the presymptomatic group, 17/49 CSF samples had quantifiable levels of cryptic HDGFL2 (range=0.10-236.7 ng/mL). The symptomatic group had the highest levels of cryptic HDGFL2 (mean=11.6, SD=29.8), the presymptomatic group had the second highest levels (mean=7.6, SD-34.1), and the control group had the least (mean=0.007, SD=0.03). CSF of 14 sporadic ALS cases from Biogen and the Northeast Amyotrophic Lateral Sclerosis Consortium (NEALS) and 17 additional symptomatic C9ORF72-mutation carriers from NEALS were also assayed. All sporadic ALS CSF had been previously thawed one time before MSD analysis. Of the 17 additional symptomatic C9ORF72 samples, 4 had been previously thawed one time while 13 had never been thawed. All CSF from healthy controls and presymptomatic and symptomatic C9ORF72-mutation carriers in the DIALS cohort had not been previously thawed before MSD analysis. All CSF from presymptomatic and symptomatic C9ORF72-mutation carriers part of the NIA cohort had been previously thawed one time.

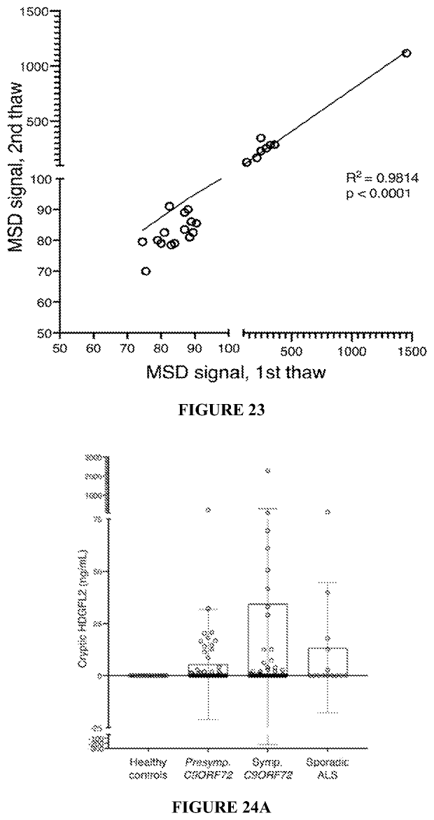

Due to the differences in freeze-thaw cycles between different samples, the effect that one freeze-thaw cycle would have on cryptic HDGFL2 measurements were analyzed. Twenty-three C9ORF72-mutation carrier CSF samples were assayed both upon first thaw and after one freeze-thaw cycle. Normalized MSD signals were slightly decreased after one freeze-thaw cycle, but relative signal levels were preserved (simple linear regression, y=0.607x+26.53, R²=0.9814, p<0.0001; FIG. 23). Twenty-three C9ORF72-mutation carrier CSF samples were assayed both upon first thaw and after one freeze-thaw cycle. Normalized MSD signals were slightly decreased for the previously thawed samples, but relative signal levels were preserved (simple linear regression, y=0.607x+26.53, R²=0.9814, p<0.0001). Previously thawed samples were normalized according to this regression model in order to compare all CSF samples.

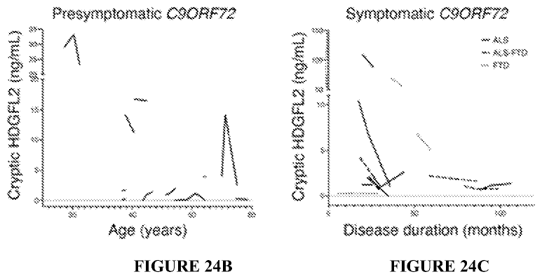

Comparing all 179 normalized CSF samples, symptomatic C9ORF72-mutation carriers had the highest cryptic HDGFL2 levels (mean=45.2 ng/mL, SD=281.9) and sporadic ALS had the next highest levels (mean=22.4 ng/mL, SD=60.5), followed by presymptomatic C9ORF72-mutation carriers (mean-6.1 ng/mL, SD=26.7; FIG. 24A). Healthy controls had the lowest levels of cryptic HDGFL2 (mean=0.007 ng/ml, SD=0.03; FIG. 24A).

### Example 9

### Csf Cryptic Hdgfl2 Levels Tend to Decrease During Symptomatic Disease Progression

Prior to the assay's current optimization, CSF of the NIA cohort were previously analyzed with the MSD assay utilizing a rabbit primary detection antibody and a sulfo-tag species-specific secondary detection antibody (FIG. 10A). This data, which had a greater dynamic range despite less specificity, suggested that cryptic HDGFL2 levels tend to be higher earlier in disease (FIGS. 10B-10C). With the current optimized assay, longitudinal CSF samples of both presymptomatic (FIG. 24BB) and symptomatic C9ORF72-mutation carriers (FIG. 24C) were analyzed elucidate the dynamics of cryptic HDGFL2 in CSF.

In 9 of 13 symptomatic C9ORF72-mutation carriers who had detectable levels of cryptic HDGFL2, cryptic HDGFL2 levels decreased with later timepoints of CSF collection. In 2 of the 13 individuals, cryptic HDGFL2 levels did not change. In one, cryptic HDGFL2 increased slightly with subsequent collections, and in another, cryptic HDGFL2 decreased from the first to the second sample collection but increased from the second to the third (FIG. 24C).

In the 11 presymptomatic C9ORF72-mutation carriers who had detectable levels of cryptic HDGFL2, cryptic HDGFL2 levels did not display such a unidirectional dynamic. In 4 of the individuals, cryptic HDGFL2 decreased longitudinally, while in another 4 cryptic HDGFL2 increased. In 3 of the 6 presymptomatic C9ORF72-mutation carriers in this group who had at least 3 longitudinal timepoints of CSF, cryptic HDGFL2 increased from the first to the second collection and decreased from the second to the third (FIG. 24B).

Taken together, these data suggest that CSF cryptic HDGFL2 levels tend to decrease during symptomatic disease and may peak presymptomatically, a trend that is in the direction opposite to that of phosphorylated neurofilament heavy (pNfH) and neurofilament light (NfL) chains (FIG. 10D). As the preliminary data indicate that a trend towards higher levels of cryptic HDGFL2 is observed during earlier-stage disease, it was hypothesize that while CSF neurofilament subunits rise during the prodromal phase and remain elevated in symptomatic disease, CSF cryptic HDGFL2 would decrease after its peak, which may occur during the prodromal stage. Thus, a ratio of pNfH or NfL to cryptic HDGFL2 of less than 1 or greater than 1 would indicate, respectively, pre-symptomatic or symptomatic phase of disease. Thus, the analysis of cryptic exon accumulation in CSF of numerous C9ORF72-linked individuals allowed not only to establish loss of TDP-43 splicing repression occurring at a presymptomatic stage, but also to discern the dynamic nature of TDP-43 cryptic proteins. it is proposed that cryptic HDGFL2 levels may peak presymptomatically in some individuals and decrease as disease progresses, so measuring CSF cryptic HDGFL2 may provide the ability to predict phenoconversion in familial ALS. While further study of individuals with sporadic ALS is warranted, these findings suggest that cryptic exon-encoded peptides could serve as biomarkers to facilitate earlier diagnosis of ALS, which is currently delayed by the need to rule out mimic conditions. Such early-stage biomarkers that can identify ALS-related pathomechanisms are currently critically lacking. Earlier diagnosis would permit prompt therapeutic intervention with a greater chance of success.

### REFERENCES

Barmada, S. J. et al (2010). Cytoplasmic mislocalization of TDP-43 is toxic to neurons and enhanced by a mutation associated with familial amyotrophic lateral sclerosis. The Journal of neuroscience: the official journal of the Society for Neuroscience, 30 (2), 639-649. doi.org/10.1523/JNEUROSCI.4988-09.2010
Brettschneider, J. et al (2013). Stages of pTDP-43 pathology in amyotrophic lateral sclerosis. Annals of Neurology, 74 (1), 20-38. doi.org/10.1002/ana.23937
Brown, A.-L. et al. (2022). TDP-43 loss and ALS risk SNPs drive mis-splicing and depletion of UNC13A. Nature, 603 (7899), 131-137 doi.org/10.1038/s41586-022-04436-3
Donde, A. et al. (2019). Splicing repression is a major function of TDP-43 in motor neurons. Acta Neuropathologica, 138, 813-826. doi.org/10.1007/s00401-019-01041-8).
Klim, J. R. et al (2019). ALS-implicated protein TDP-43 sustains levels of STMN2, a mediator of motor neuron growth and repair. Nature Neuroscience, 22 (2), 167-179. doi.org/10.1038/s41593-018-0300-4
Ling, J. P. et al (2015). TDP-43 repression of nonconserved cryptic exons is compromised in ALS-FTD. Science, 349 (6248), 650-655. https://doi.org/10.1126/science.aab0983
Ma, X. R. et al (2022). TDP-43 represses cryptic exon inclusion in the FTD-ALS gene UNC13A. Nature, 603 (7899), 124-130. doi.org/10.1038/s41586-022-04424-7
Melamed, Z. et al (2019). Premature polyadenylation-mediated loss of stathmin-2 is a hallmark of TDP-43-dependent neurodegeneration. Nature neuroscience, 22 (2), 180-190. doi.org/10.1038/s41593-018-0293-z
Neumann, M. et al (2006). Ubiquitinated TDP-43 in frontotemporal lobar degeneration and amyotrophic lateral sclerosis. Science, 314 (5796), 130-133. doi.org/10.1126/science.1134108
Prudencio, M. et al (2020). Truncated stathmin-2 is a marker of TDP-43 pathology in frontotemporal dementia. The Journal of clinical investigation, 130 (11), 6080-6092. doi.org/10.1172/JCI139741
Rowland, L. P., & Shneider, N. A. (2001). Amyotrophic lateral sclerosis. The New England journal of medicine, 344 (22), 1688-1700. https://doi.org/10.1056/NEJM200105313442207
Sun, M. et al (2017). Cryptic exon incorporation occurs in Alzheimer's brain lacking TDP-43 inclusion but exhibiting nuclear clearance of TDP-43. Acta Neuropathologica, 133 (6), 923-9331. doi.org/10.1007/s00401-017-1701-2
Traynor, B. J. et al (2000). Amyotrophic lateral sclerosis mimic syndromes: a population-based study. Archives of neurology, 57 (1), 109-113. https://doi.org/10.1001/archneur.57.1.109
Zhang, Y. J. et al (2009). Aberrant cleavage of TDP-43 enhances aggregation and cellular toxicity. Proceedings of the National Academy of Sciences of the United States of America, 106 (18), 7607-7612. doi.org/10.1073/pnas.0900688106

Although the invention has been described with reference to the above examples, it will be understood that modifications and variations are encompassed within the spirit and scope of the invention. Accordingly, the invention is limited only by the following claims.

1. A method of detecting TDP-43 loss of function in a subject comprising contacting a sample from the subject with an antibody or binding fragment thereof which specifically binds to a cryptic exon-encoded neoepitope,
wherein the cryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2),
thereby detecting TDP-43 loss of function in the subject.

2. The method of claim 1, wherein detecting TDP-43 loss of function comprises detecting a cryptic exon-encoded neoepitope in the sample from the subject.

3. The method of claim 2, wherein the sample is a biological fluid.

4. The method of claim 3, wherein the biological fluid is selected from the group consisting of blood, cerebrospinal fluid (CSF), saliva, sputum, urine or another biofluid.

5. The method of claim 1, wherein the cryptic exon-encoded neoepitope is within HDGFL2.

6. A method of detecting and/or diagnosing a TDP-43-associated disease in a subject comprising detecting TDP-43 loss of function in the subject using the method of claim 1,
thereby detecting or diagnosing the TDP-43-associated disease in the subject.

7. (canceled)

8. The method of claim 6, further comprising detecting one or more additional TDP-43-associated biomarkers in the sample selected from the group consisting of neurofilament (NF), tau, amyloid-β, α-synuclein, and combinations thereof.

9. (canceled)

10. The method of claim 8, wherein the TDP-43-associated disease is selected from the group consisting of Alzheimer's disease (AD), amyotrophic lateral sclerosis (ALS), frontotemporal lobar degeneration (FTLD), inclusion body myositis (IBM), primary age-related tauopathy (PART)/Neurofibrillary tangle-predominant senile dementia, chronic traumatic encephalopathy (CTE), progressive supranuclear palsy (PSP), corticobasal degeneration (CBD), frontotemporal dementia and parkinsonism linked to chromosome 17 (FTDP-17), lytico-bodig disease (Parkinson-dementia complex of Guam), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, chronic traumatic encephalopathy, limbic-predominant age-related TDP-43 encephalopathy (LATE), multiple sclerosis (MS) and TDP-43 encephalopathy.

11. (canceled)

12. The method of claim 6, wherein the TDP-43 associated disease is an early stage of the disease or in a pre-symptomatic phase of the disease.

13. The method of claim 6, wherein the cryptic exon-encoded neoepitope is within HDGFL2.

14. A method of detecting cryptic exon-encoded neoepitopes in a sample from a subject,
comprising contacting the sample with an antibody or binding fragment thereof which specifically binds to a cryptic exon-encoded neoepitope,
thereby detecting cryptic exon-encoded neoepitopes in the subject.

15. The method of claim 14, wherein the cryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2).

16. (canceled)

17. The method of claim 14, wherein an increase in the detection of a cryptic exon-encoded neoepitope or an increase in a number of cryptic exon-encoded neoepitopes detected in the sample in a first detection as compared to a second detection is indicative of disease progression and/or of an absence of response to the therapy.

18. The method of claim 14, wherein a decrease in the detection of a cryptic exon-encoded neoepitope or a decrease in a number of cryptic exon-encoded neoepitopes detected in the sample in a first detection as compared to a second detection is indicative of an absence of disease progression, a disease regression, and/or of a response to the therapy.

19. The method of claim 14, wherein the cryptic exon-encoded neoepitope is within HDGFL2.

20. A method of selecting a patient for enrollment in a clinical trial comprising detecting cryptic exon-encoded neoepitopes in a sample from the subject using the method of claim 14,
thereby selecting the patient for enrollment in the clinical trial.

21-24. (canceled)

25. A method of predicting pheno-conversion of a TDP-43 associated disease in a subject comprising determining a ratio of a TDP-43-associated biomarker to a cryptic exon-encoded neoepitope in a sample from the subject using the method of claim 14,
thereby predicting pheno-conversion in the subject.

26-34. (canceled)

35. A kit comprising:
a) the antibody or binding fragment thereof of claim 37; and
b) instructions to use the antibody of a) to detect TDP-43 loss of function in a sample,
wherein the cryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2).

36. The kit of claim 35, further comprising an antibody or binding fragment thereof which specifically bind to phosphorylated neurofilament heavy chain (pNFH).

37. An antibody or binding fragment thereof which specifically binds to a cryptic exon-encoded neoepitope, wherein the cryptic exon-encoded neoepitope is an epitope resulting from a splicing incorporation of an exon normally repressed by TDP-43, and
wherein the cryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2).

38. The antibody of claim 37, wherein the splicing incorporation of an exon normally repressed by TDP-43 results from a TDP-43 loss of function.

39. The antibody of claim 38, wherein TDP-43 loss of function generates exons fused-in-frame with a translational reading frame to produce neoepitopes.

40. A method of detecting TDP-43 loss of function in a subject comprising detecting in a sample from the subject the presence of a cryptic exon-encoded neoepitope using the method of claim 14,
wherein the cryptic exon-encoded neoepitope is an epitope resulting from a splicing incorporation of an exon normally repressed by TDP-43, and
wherein the cryptic exon-encoded neoepitope is within (i) hepatoma derived growth factor like 2 (HDGFL2), (ii) actin-like protein 6B (ACTL6B), (iii) Rho GTPase-activating protein 32 (ARHGAP32), (iv) band 4.1-like protein 4A (EPB41L4A), (v) sodium/potassium/calcium exchanger 3 (SLC24A3), (vi) cysteine dioxygenase type 1 (CDO1), (vii) agrin (AGRN), (viii) IgLON Family Member 5 (IGLON5), (ix) dynamin-1 (DNM1), (x) alanyl-TRNA synthetase 1 (AARS1), (xi) peroxidasin (PXDN), or (xii) N-terminal EF-hand calcium-binding protein 2 (NECAB2),
thereby detecting TDP-43 loss of function in the subject.

41-43. (canceled)

44. The method of claim 2, wherein detecting the presence of a cryptic exon-encoded neoepitope is by enzyme-linked immunosorbent assay (ELISA), protein immunoprecipitation, immunoelectrophoresis, western blot, protein immunostaining, high-performance liquid chromatography (HPLC), or liquid chromatography-mass spectrometry (LC/MS).

45. A method of monitoring a TDP-43-associated disease progression and/or response to a TDP-43-associated disease therapy in a subject comprising detecting cryptic exon-encoded neoepitopes in a sample from a subject using the method of claim 14,
thereby monitoring the TDP-43-associated disease progression and/or response to therapy in the subject.
